SEQUENCE LISTING (1 sequence)

SEQ ID NO: 1 - protein, 32 aa, mammalian
VPTARRPLSLLYYDRDSNIVKTDIPDMVVEAC

(19) United States
(12) Patent Application Publication
(10) Pub. No.: US 2006/0172347 A1
(43) Pub. Date: Aug. 3, 2006

(54) Method of diagnosis, treatment and useful agents for conditions characterised by modulation in the level of activin ssc

(75) Inventors: Sally Mellor, Sandringham (AU); Gail Risbridger, East Malvern (AU); Emma Ball, South Yarra (AU); Hong Wang, Mount Waverley (AU); Catriona McClean, Hawthorn (AU); John Pedersen, Hawthorn (AU); Anne O'Connor, Brunswick East (AU); Mark Cranfield, Oxford (GB); Nigel Groome, Oxford (GB)

Correspondence Address: SCULLY, SCOTT, MURPHY & PRESSER, 400 GARDEN CITY PLAZA, SUITE 300, GARDEN CITY, NY, 11530, US

(73) Assignee: Monash University, Victoria (AU)

(21) Appl. No.: 10/538,431

(22) PCT Filed: Dec. 12, 2003
(86) PCT No.: PCT/AU03/01665

§ 371 Date: Jan. 9, 2006

(30) Foreign Application Priority Data
Dec. 12, 2002 (AU) 2002953327

(51) Int. Cl. G01N 33/574

(52) U.S. Cl. 435/7.23

(57) ABSTRACT

The present invention relates generally to a method of diagnosing, predicting or monitoring the development or progress of a condition characterised by modulation in the level of activin expression and, more particularly, a method of diagnosing, predicting or monitoring the development or progress of a condition characterised by modulation in the level of expression of activin bc subunit. The present invention still further provides methods for the therapeutic or prophylactic treatment of conditions characterised by aberrant, unwanted or otherwise inappropriate activin expression, for example, conditions characterised by over-expression or underexpression of activin and most particularly, conditions characterised by overexpression or underexpression of activin bc subunit. A further aspect of the present invention extends to agents for use in the methods of the present invention.

### FIELD OF THE INVENTION

The present invention relates generally to a method of diagnosing, predicting or monitoring the development or progress of a condition characterised by modulation in the level of activin expression and, more particularly, a method of diagnosing, predicting or monitoring the development or progress of a condition characterised by modulation in the level of expression of activin β_{c} subunit. The present invention still further provides methods for the therapeutic or prophylactic treatment of conditions characterised by aberrant, unwanted or otherwise inappropriate activin expression, for example, conditions characterised by overexpression or underexpression of activin and most particularly, conditions characterised by overexpression or underexpression of activin β_{c} subunit. A further aspect of the present invention extends to agents for use in the methods of the present invention.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Activins, are members of the TGF-β superfamily that have diverse roles as potent growth and differentiation factors in many organs and tissues. Activins are homo- or heterodimers of activin P subunits, such as β_{A}, β_{B}, β_{C}, β_{D} or β_{E} that form activin dimer ligands. The activin family encompasses disulfide-linked dimeric proteins characterized by a conserved cysteine-knot motif. Activin A (β_{A}-β_{A}) was originally isolated in ovarian follicular fluid as a stimulator of FSH secretion. Nevertheless, it is now recognised that activins such as activin A (β_{A}-β_{A}), activin B (β_{B}-β_{B}), and activin AB (β_{A}-β_{B}) have a range of biological activities that include mesoderm induction in Xenopus laevis embryos, immune suppression, bone growth, nerve cell survival, wound healing, tumourogenesis and tissue differentiation in pancreas, kidney and heart (Luisi et al, 2001. Eur J Endocrinol 145:225-36, McDowell et al, 1999. Semin Cell Dev Biol 10:311-7, de Kretser et al, 1999. J Endocrinol 161:195-8, Hubner et al, 1999. Histol Histopathol 14:295-304). Some activin family members appear to be involved in differentiation and control of proliferation. Examples of activin dimer ligands involved in these processes include activin A (β_{A}-β_{A}), activin B (β_{B}-β_{B}), and heterodimer activin AB (β_{A}-β_{B}). More recently, an activin β_{C} subunit, together with activin β_{D} and β_{E} subunits, have been identified, which form a new subset of activin β subunits. Activin β_{c}-β_{c} forms the activin C homodimer (Kron et al, 1998, J Virol Methods 72:9-14).

The activin ≈_{c} subunit was cloned from mouse (Lau et al, 1996, Biochim Biophys Acta 1307:145-8) and human liver (Hotten G et al, 1995, Biochem Biophys Res Commun 206:608-13)Activin β_{D} has been cloned from Xenopus. Microinjection of β_{D} cDNA induces mesoderm induction, however no mammalian equivalent has been identified (Oda et al, 1995, Biochem Biophys Res Commun 210:581-8). Activin β_{E} subunit was cloned from mouse liver and found to be expressed in rat liver and lung (O'Bryan et al, 2000, J Mol Endocrinol 24:409-18). Zhang and others demonstrated differences in β_{A} and β_{C} mRNA regulation following rat partial hepatectomy and proposed that activin β_{C} was a liver chalone (Esquela et al, 1997, Biochem Biophys Res Commun 235:553-6, Zhang et al,1997, Endocr J 44:759-64) However, no biological role for activins D (β_{D}β_{D} homodimer)or E (β_{E}β_{E} homodimer) has been established.

Similarly, neither the activin β_{C} subunit nor activin C have been implicated in any of the above mentioned biological processes. In addition, no biological activity has been determined for activin β_{C} or activin C (β_{C}-β_{C}). For example, Groome et al, (2001, J. Mol. Cell. Endo. 180: 73-77) refer to the “continuing failure of activins C . . . to display a bioactivity”, Lau et al, (2000, Mol Cel Biol, 20 (16):6127-37) state that “activin betaC . . . are not essential for either embryonic development or liver function” and Chang et al (2001, Mol Cell Endocrinol. Jun 30;180(1-2):39-46) state that activin betaC is “not essential for liver growth, differentiation and regeneration”.

Activin β_{C} subunit has frequently been referred to in the literature as a “liver specific activin”. Fang et al. described activin β_{C} expression in adult mice as “a unique liver-restricted pattern” (Fang J et al, 1997, Biochem Biophys Res Commun. 231 (3):655-61) Furthermore, Lau et al. stated that activin β_{C} exhibited a “highly restricted tissue expression pattern” in the mammalian liver (Lau et al, 2000. supra) while Schmitt et al. found that “the inhibin/activin β_{C} gene is predominantly expressed in adult mouse liver” and that the expression level in liver was “specific and high” (Schmitt et al, 1996, Genomics 32:358-66) In addition, Chang et al. described activin β_{C} as being “expressed primarily in the liver in the adult” and having a “highly restricted tissue-specific expression pattern” (Chang et al, 2001, Mol Cell Endocrinol. Jun 30;180(1-2):39-46). Kron et al. stated that “the β_{C} subunit is exclusively expressed in liver tissue” (Kron et al, 1998, supra).

Activin signal transduction is initiated by ligand binding inducing the formation of a heteromeric receptor complex of type I and II transmembrane serine/threonine kinase receptors. Activin binding to ActRII or IIB, results in recruitment and phosphorylation of type I receptor ActRI, thereby initiating the phosphorylation of downstream signalling proteins, the Smad (Sma- and Mad-related) proteins. Following phosphorylation, Smad2 and 3 (receptor-regulated Smads), form a heteromeric complex with Smad4 (co-Smad) and translocate from the cytoplasm to the nucleus (Lebrun et al, 1999, Mol Endocrinol 13:15-23; Wrana and Attisano, 2000, Cytokine Growth Factor Rev 11:5-13; Pangas et al, 2000, Trends Endocrinol Metab 11:309-314.). Interaction of Smad proteins with either transcription factors or DNA-binding elements regulate appropriate gene expression. For example, in Xenopus, the DNA binding transcription factor, forkhead activin signal transducer-I (FAST-I) binds to the Smad2 and Smad4 complex to activate the activin response element (ARE) on the Xenopus Mix.2 promoter (Chen et al, 1996 Nature 383:691-6; Chen et al, 1997, Nature 389:85-9). It is not known if activin β_{C} and β_{E} subunits transduce a signal through the above activin receptors or if they have their own receptors.

Little is known about the presence, significance or function of activin β_{C} subunit protein in tissues or organs. In addition, little is known about the formation of activin dimers and the regulation of activin dimer formation. In particular, the regulation of dimerisation of activin subunits β_{A}, β_{B}, β_{C}, β_{D} or ≈_{E}, or combinations thereof.

Accordingly, there is a need to elucidate the role of activin C and/or the activin β_{C} subunit in the context of what is currently known of the functionality of the activin subunit molecules in their monomeric or dimeric forms. This would enable both the refinement of existing diagnostic and therapeutic treatment regimes and the development of new regimes.

In work leading up to the present invention it has been determined that changed levels of activin β_{C} subunit, relative to normal levels in either monomeric or dimeric form are associated with the onset of a neoplastic condition. Accordingly, these findings have now facilitated the development of an assay directed to diagnosing and/or monitoring disease conditions characterised by modulation of activin β_{C} levels, such as neoplastic conditions. Further there are also now provided methods for the therapeutic and/or prophylactic treatment of these conditions.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source.

The subject specification contains nucleotide sequence information prepared using the programme Patent In Version 3. 1, presented herein after the bibliography. Each amino acid sequence is identified in the sequence listing by the numeric indicator <210> followed by the sequence identifier (eg. <210>1, <210>2, etc). The length, type of sequence (protein, etc) and source organism for each amino acid sequence is indicated by information provided in the numeric indicator fields <211>, <212>and <213>, respectively. Amino acid sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing.

One aspect of the present invention is directed to a method of detecting in a mammal the onset, or predisposition to the onset, of a condition characterised by modulation of the level or bioactivity of activin β_{C}, which level is modulated relative to normal levels, said method comprising screening for the level of activin β_{C} protein and/or gene expression in a biological sample derived from said mammal.

Another aspect of the present invention is directed to a method of detecting the onset, or predisposition to the onset, of a condition characterised by an increase or decrease in the level or bioactivity of activin β_{C} in a mammal, relative to normal levels, said method comprising screening for the level of activin β_{C} protein and/or gene expression in a biological sample derived from said mammal.

Still another aspect of the present invention is directed to a method of monitoring for the onset or progression of a condition characterised by modulation of the level or bioactivity of activin β_{C} in a mammal, which level is modulated relative to normal levels, said method comprising screening for the level of activin β_{C} protein and/or gene expression in a biological sample derived from said mammal.

Yet still another aspect of the present invention is directed to a method of monitoring for the onset or progression of a condition characterised by an increase or decrease in the level or bioactivity of activin β_{C} in a mammal, relative to normal levels, said method comprising screening for the level of activin β_{C} protein and/or gene expression in a biological sample derived from said mammal.

In a further aspect there is provided a method of detecting the onset, or predisposition to the onset, of a condition characterised by modulation of the level of activin β_{C} in a mammal, which level is modulated relative to normal levels, said method comprising:
(a) contacting a first antibody that recognises an epitope of a first activin β subunit with a biological sample derived from said mammal;
(b) allowing the first antibody to bind to said first activin p subunit in said sample;
(c) washing said sample to substantially remove unbound material;
(d) contacting said sample with a second antibody that recognises an epitope of a second activin β subunit, wherein the second antibody is tagged with a labelling agent; and
(e) detecting the labelling agent to identify an activin β_{C} subunit dimer in said sample, wherein the first or second antibody recognises an epitope of an activin β_{C} subunit.

Yet another aspect of the present invention is directed to a composition when used to detect the onset, or predisposition to the onset, of a condition characterised by modulation of the level or bioactiviy of activin β_{c}, in accordance with the methods hereinbefore described, said composition comprising an activin β_{C} detection means

Another further aspect of the present invention provides a composition when used to detect the onset, or predisposition to the onset, of a condition characterised by modulation of the level or bioactiviy of activin β_{c}, wherein said composition comprises an antibody directed to an epitope of an activin β_{C} subunit together with a suitable diluent, excipient or carrier. Yet another aspect of the present invention provides a diagnostic kit for use in detecting the onset, or predisposition to the onset, of a condition characterised by modulation of the level or bioactiviy of activin β_{C} subunit, said kit comprising an activin β_{C} subunit protein and/or encoding nucleic acid detection means in a first compartment and reagents useful for facilitating detection by said detection means in a second compartment. Further compartments may also be included, for example, to include means for facilitating the collection and storage of a biological sample.

Another aspect of the present invention is directed to a method of modulating the abnormal growth of a cell, said method comprising modulating the level or bioactivity of activin β_{C} subunit.

More particularly, the present invention is directed to a method of modulating the abnormal growth of a cell, said method comprising modulating the level or bioactivity of activin β_{C} subunit wherein up-regulating activin β_{C} subunit levels or bioactivity to a functionally effective level induces said abnormal growth and down-regulating activin β_{C} subunit levels or bioactivity to a functionally ineffective level inhibits said abnormal growth.

Even more particularly, the present invention is directed to a method of modulating the growth of a cell, said method comprising modulating the level or bioactivity of activin β_{C} subunit wherein down-regulating activin β_{C} subunit levels or bioactivity to a functionally ineffective level induces said cell growth and up-regulating activin β_{C} subunit levels or bioactivity to a functionally effective level inhibits said cell growth.

In still another further aspect there is provided a method of down-regulating the growth of a neoplastic cell in a mammal, said method comprising administering to said mammal an effective amount of an agent for a time and under conditions sufficient to induce a functionally ineffective level of activin β_{C} subunit.

In still another further aspect there is provided a method of down-regulating the growth of a neoplastic cell in a mammal, said method comprising administering to said mammal an effective amount of an agent for a time and under conditions sufficient to induce a functionally effective level of activin β_{C} subunit.

The present invention contemplates a method of therapeutically and/or prophylactically treating a condition, or a predisposition to the development of a condition, characterised by an aberrant, unwanted or otherwise inappropriate level or bioactivity of activin β_{C} subunit in a mammal, said method comprising modulating the level of activin β_{C} subunit in said mammal.

The present invention preferably contemplates a method of therapeutically and/or prophylactically treating a neoplastic condition, or a predisposition to the development of a neoplastic condition, said method comprising modulating the level or bioactivity of activin β_{C} subunit wherein down-regulating said activin β_{C} subunit level to a functionally ineffective level inhibits abnormal cell growth.

The present invention preferably contemplates a method of therapeutically and/or prophylactically treating a neoplastic condition, or a predisposition to the development of a neoplastic condition, said method comprising modulating the level or bioactivity of activin β_{C} subunit wherein up-regulating said activin β_{C} subunit level to a functionally effective level inhibits abnormal cell growth.

Another aspect of the present invention relates to the use of an agent capable of modulating the functionally effective level of activin β_{C} subunit in the manufacture of a medicament for the treatment of a condition characterised by an aberrant, unwanted or otherwise inappropriate level of activin β_{C} subunit.

In another aspect the present invention relates to the use of an agent as hereinbefore described in the manufacture of a medicament for the regulation of the abnormal growth of a cell wherein down-regulating activin β_{C} subunit to a functionally ineffective level inhibits abnormal growth.

In another aspect the present invention relates to the use of an agent as hereinbefore described in the manufacture of a medicament for the regulation of the abnormal growth of a cell wherein up-regulating activin β_{C} subunit to a functionally effective level inhibits abnormal growth.

In yet another further aspect, the present invention contemplates a pharmaceutical composition comprising the modulatory agent as hereinbefore defined together with one or more pharmaceutically acceptable carriers and/or diluents.

### BRIEF DESCRIPTION OF THE DRAWINGS

FIG. 1 shows immunolocalisation of activin β_{C} subunit protein in human adrenal and thyroid gland and following the development of cancer in these organs. Insets show low power view of whole tissue section.

(A) The cortex of the adrenal gland displays an isolated nuclear (arrow) staining pattern for activin β_{C} subunit protein. Weak positive and strong positive (arrowhead) cytoplasmic staining was also observed in the adrenal medulla. (B) Tissue from a patient with adrenal cortical carcinoma displayed predominantly strong nuclear (arrow) activin β_{C} subunit immunolocalisation, however cytoplasmic (arrowhead) staining was also observed. (C) The follicles of the thyroid gland display intermittent immunolocalisation for activin β_{C} subunit protein. Predominantly, epithelial cells of the follicles display no staining (arrow) for the activin β_{C} subunit, however some epithelial cells have cytoplasmic localisation (arrowhead). (D) In contrast, a patient with thyroid minimally invasive follicular carcinoma displayed strong activin β_{C} subunit staining in the cytoplasm (arrow). (E) In addition, a patient with papillary carcinoma of the thyroid gland immunolocalised strongly to the nuclei (arrow) and was less intense in the cytoplasm (arrowhead).

FIG. 2 shows immunolocalisation of activin β_{C} subunit protein in normal human digestive tissues (stomach, rectum, colon) and following the development of adenocarcimoma. Insets show low power view of whole tissue section.

(A,B) Activin β_{C} subunit protein was localised to epithelial cells (arrow) of the human stomach, however the staining pattern was variable. Smooth muscle cells and macrophages displayed variable staining. (C) In a patient with moderately differentiated stomach adenocarcinoma, a pattern of predominantly cytoplasmic staining (arrow) was observed. (D) In contrast, a patient with poorly differentiated stomach adenocarcinoma displayed strong nuclear (arrow) staining, with less intense cytoplasmic (arrowhead) staining. (E) Similarly, in patients with stomach adenocarcinoma that metastasised to the lymph node, strong nuclear staining (arrow) was observed. (F) The benign colon displays strong activin

β_{C} subunit protein immunolocalisation in some secretory epithelial cells (arrow) and smooth muscle cells. Nuclear staining was observed intermittently (G) Tissue from a patient with adenocarcioma of the colon displayed strong nuclear (arrow) and cytoplasmic (arrowhead) staining. (H) The normal rectum displayed both cytoplasmic and nuclear staining of the surface epithelium (I) Rectal adenocarcinoma displayed both nuclear (arrow) and cytoplasmic (arrowhead) staining however this is not observed in all tumour cells.

FIG. 3 shows immunolocalisation of activin β_{C} subunit protein in normal, urinary bladder, skin, breast, lymph node and following the development of cancer in these tissues. Inserts show low power view of whole tissue section.

(A) The transitional epithelium of the urinary bladder immunolocalises activin β_{C} subunit protein, both the cytoplasm and some nuclei. Intermittent smooth muscle cells also display positive staining. (B) Urinary bladder poorly differentiated carcinoma strongly immunolocalises the nuclei of these tumour cells, however the cytoplasm also shows positive staining. (C) The skin immunolocalises the activin β_{C} subunit in the cytoplasm of keratinocytes as well as some nuclei, hair follicles, and blood vessels. (D) In tissue from a patient with skin squamous cell carcinoma, activin β_{C} subunit protein strongly immunolocalises to the nuclei of the tumour cells, however the cytoplasm is also postive. (E) Normal breast epithelium immunolocalises activin β_{C} subunit protein. Myoepithelial cells displayed both positive (arrow) and negative staining (arrowhead), however the secretory epithelial cells showed strong cytoplasmic localisation (asterisk). (F) In contrast, patients with breast residual infiltrating duct carcinoma display strong nuclear staining, as well as cytoplasmic localisation. (G) Breast infiltrating lobular carcinoma tissue also displayed predominantly nuclear localisation associated with weak cytoplasmic staining. (H) Tissue from a patient with breast papillary carcinoma displayed strong nuclear and cytoplasmic staining. (I) The normal lymph node tissue immunolocalised activin β_{C} subunit protein in the stromal tissue (arrow) surrounding the lymphyocytes. However the lymphocytes themselves were negative for the activin β_{C} subunit (arrowhead). (J) Tissue from a patient with lymphoma displayed strong nuclear staining, however not all nuclei were positive. Some tumour cells displayed cytoplasmic immunolocalisation.

FIG. 4 shows immunolocalisation of activin β_{C} subunit protein in normal human salivary gland, bone, nasal cavity and following the development of cancer in these tissues. Insets show low power view of whole tissue section.

(A) In the salivary gland, cytoplasmic localisation for activin β_{C} subunit protein is observed in the ducts (arrow), serous cells (arrowhead), mucous cells (asterisk) and nerves of this organ. (B) In a patient with a Warthin tumour of the parotid gland, cytoplasmic and some nuclei staining is observed in the tumour cells. (C) Tissue from a patient with carcinoma of the submandibular gland immunolocalises activin β_{C} subunit protein to the cytoplasm and the nuclei of these tumour cells. (D) Tissue from a patient with low grade chondrosarcoma, activin β_{C} subunit protein displayed focal nuclear localisation of chondrocytes. (E) In contrast, tissue from a patient with bone osteosarcoma shows predominant positive staining in the cytoplasm, however there is also some nuclear staining. (F) Both strong cytoplasmic and nuclear staining is observed in a patient with bone giant cell tumour. (G) Tissue from the normal nasal cavity displays activin β_{C} subunit immunolocalisation in the epithelium of the nasal mucosa. Specifically in both the basal cells (the proliferative area of the epithelium), and more predominantly localised in the secretory epithelial cells. (H) In tissue from a patient with inverted papilloma of the nasal cavity, cytoplasmic and nuclear localisation was observed in the tumour cells.

FIG. 5 shows immunolocalisation of activin β_{C} subunit protein in normal human stomach and duodenum and following the development of cancer in these tissues. Insets show low power view of whole tissue section.

Normal stomach tissue immunolocalises activin β_{C} subunit protein in both the glands and smooth muscle, however this localisation is intermittent with both positive and negative staining. (A) In normal tissue, glands displayed both nuclear and cytoplasmic immunolocalisation but staining was non-uniform. (B) In the antrum of the stomach displays immunolocalisation in both the mucosa and muscle layers, but not all cells are positive. For example, the gastric surface displays cytoplasmic localisation. (C) The duodenum immunolocalises activin β_{C} subunit protein in both the mucosal and smooth muscle cell layer. Not all cell types are positive and localisation is non-uniform. In the luminal surface secretory cells, some cells that display activin β_{C} subunit localisation in the cytoplasm, while others have nuclear staining in the deeper layers of the mucosa. (D) Tissue from a patient with moderately differentiated stomach adenocarcinoma displayed predominantly cytoplasmic activin β_{C} subunit immunolocalisation. (E) In contrast, both nuclear and cytoplasmic immunolocalisation was observed in a patient with poorly differentiated stomach adenocarcinoma. (F) Nuclear staining was also observed in a patient with signet ring cell carcinoma of the stomach, in addition to stromal staining. (G) Tissue from lymphoma of the stomach displayed a similar pattern of staining in the nuclei of tumour cells and stromal cells. (H) Stomach carcinoma that had metastasised to the lymph node, displayed intermittent nuclear, cytoplasmic and stromal localisation.

FIG. 6 shows immunolocalisation of activin β_{C} subunit protein in normal human gallbladder and urinary bladder and following the development of cancer in these tissues. Insets show low power view of whole tissue section.

(A) In the normal gallbladder, basal and secretory cells localise the activin β_{C} subunit.

Both nuclear and cytoplasmic staining is observed in the epithelial cell layer. Smooth muscle localisation was also observed. (B) Similarly, tissue from a patient with adenocarcinoma of the gallbladder displayed both nuclear and cytoplasmic staining in the tumour cells. In addition, smooth muscle (asterisk; inset) in the vicinity of the tumour cells displayed strong activin β_{C} subunit protein localisation. (C) In tissue from the urinary bladder, the transitional epithelium immunolocalises activin β_{C} subunit protein, in a predominantly a cytoplasmic pattern, however some cells do display nuclear immunolocalisation. (D) Tissue from a patient with high grade transitional cell carcinoma of the urinary bladder, immunolocalises activin β_{C} subunit protein in a both cytoplasmic and nuclear pattern in these tumour cells. (E) In addition, poorly differentiated carcinoma cells have strong cytoplasmic and strong nuclear staining.

FIG. 7 shows immunolocalisation of activin β_{C} subunit protein in normal adrenal gland and uterine cervix and following the development of cancer in these tissues. Insets show low power view of whole tissue sections.

(A) In the cortex of the adrenal gland, activin β_{C} subunit protein is observed in the cytoplasm, however this localisation is variable with both weak and strong areas of staining. In addition, nuclear localisation in occasionally observed. (B) Tissue from a patient with cortical carcinoma of the adrenal gland displays strong cytoplasmic and nuclear staining. (C) The uterine cervix displays some nuclear staining, however not all cells are positive. Both the cytoplasm and nuclei immunolocalise the activin β_{C} subunit in squamous dysplasia. (D) Tissue from a patient with squamous cell carcinoma of the uterine cervix immunolocalises the activin β_{C} subunit protein in the cytoplasm (arrowhead) of tumour cells. Some tumour cells also display prominent nuclear (arrow) localisation.

FIG. 8 shows immunolocalisation of activin β_{C} subunit protein in the normal pancreas and esophagus and following the development of cancer in these tissues. Insets show low power view of whole tissue sections.

(A) The pancreas immunolocalises activin β_{C} subunit protein strongly in the secretory granules of the acinar cells (arrowhead) and more weakly to the islet cells (arrow). (B) Tissue from a patient with pancreatic cancer displayed stronger activin β_{C} subunit localisation in the tumour cells. Both cytoplasmic and nuclear staining was observed in the tumour cells. (C) In the esophagus, activin β_{C} subunit immunolocalisation was observed in blood vessels and some smooth muscle. However, apart from some sporadic nuclear positive cells, the epithelial layer was negative. (D) Tissue from a patient with squamous cell carcinoma, strongly localised activin β_{C} subunit protein in the cytoplasm of the tumour cells.

FIG. 9 shows immunolocalisation of activin β_{C} subunit protein in normal human thyroid and thymus and following the development of cancer in these tissues. Insets show low lower view of whole tissue sections.

(E) In the normal thyroid gland, activin β_{C} subunit protein localisation in the epithelial cells of the thyroid follicles is intermittent and the gland is predominantly negative. The positive cells may have both cytoplasmic and nuclear staining. (F) In contrast, tissue from a patient with minimally invasive follicular carcinoma of the thyroid displayed strong localisation in the cytoplasm of the tumour cells. (G) In the normal thymus, lymphocytes are negative for the activin β_{C} subunit (arrowhead), however the thymic epithelium (arrow) displays cytoplasmic and weak nuclear staining. Stromal cells (asterisk) are also positive. (H) In tissue from a patient with thymoma, the tumor cells display strong activin β_{C} subunit protein cytoplasmic localisation. The lymphocytes remain negative for activin β_{C} subunit protein with malignancy.

FIG. 10 shows immunolocalisation of activin β_{C} subunit protein in human myometrium, benign uterus and fallopian tube. Insets show low power view of whole tissue sections. (A) In the myometrium, activin β_{C} subunit protein immunolocalisation is weak or negative. (B) Tissue from a patient with leiomyoma of the uterus displayed positive staining in smooth muscle cells. Some nuclear staining was also observed. (C) The fallopian tube immunolocalised activin β_{C} subunit protein in secretory cells, some intermittent nuclear staining was also present.

FIG. 11 shows immunolocalisation of activin β_{C} subunit protein in normal human tonsil, seminal vesicle, spleen, and appendix. Insets show low power view of whole tissue sections.

(A) In the tonsil, activin β_{C} subunit protein localised to the stromal cells (arrow) but not the lymphocytes (arrowhead). (B) In the spleen, blood vessels are strongly positive (arrow), while the lymphoid aggregations (arrowhead) are negative. (C) The secretory epithelial cells of the seminal vesicle displayed both cytoplasmic (arrowhead) and nuclear (arrow) staining for the activin β_{C} subunit. Smooth muscle cells were also positive. (D) The cytoplasm of the secretory epithelial cells (arrowhead) in the appendix strongly immunolocalise activin β_{C} subunit protein, however some nuclear staining (arrow) is also observed.

FIG. 12 shows immunolocalisation of activin β_{C} subunit protein in the normal and diseased human brain. Insets show low power view of whole tissue sections.

(A) In tissue from a patient with glioblastoma, the benign region displays astroctyes that strongly immunolocalise activin β_{C} subunit protein in the cytoplasm (arrow). Reactive astrocytes are also positive. (B) In the same patient, the blood brain barrier (arrow) also strongly localises the activin β_{C} subunit. (C) The cytoplasm of glioblastoma tumour cells (arrow) are positive for activin β_{C} subunit protein. (D) Tissue from a patient with meningioma also strongly localises activin β_{C} subunit protein in the cytoplasm of the tumour cells. (E) The grey matter of the human brain displays positive staining in neuronal cells. Activin β_{C} subunit protein immunolocalises to the white matter (F), the cerebellum (G) and the pituitary gland (H) of the human brain.

FIG. 13 shows immunolocalisation of activin β_{C} subunit protein in the normal brain of the sheep and both wild type and transgenic mice that express a human Cu,Zn Superoxide Dismutase mutation resulting in neurodegenerative disease.

Both the transgenic mice brain (A) and wild type (B) mouse brain display activin β_{C} subunit localisation in cerebellum. The molecular layer strongly displays activin β_{C} subunit protein (arrow), the granular layer displays less staining (asterisk) and the Purkinje cells (arrowhead) are negative. (C) The endocrine cells (arrow) of the sheep pituitary gland immunolocalise activin β_{C} subunit protein. (D) In the pre-optic area of the sheep brain, neuronal cells with axon processes (arrow) localise the activin β_{C} subunit. (E) In the sheep hypothalamus neuronal cells (arrow) display activin β_{C} subunit protein localisation.

FIG. 14 shows immunolocalisation of activin β_{C} subunit protein in malignant human skin, larynx, tongue, small intestine and disorders of the appendix and soft tissue.

(A) Tissue from a patient with melanoma displays activin β_{C} subunit localisation in the cytoplasm and nuclei of tumour cells. (B) In a patient with pseudomyxoma of the appendix, cytoplasmic and some nuclear staining is observed. (C) Activin β_{C} subunit protein immunolocalises to the cytoplasm and some nuclei in a patient with neurofibromatosis of the soft tissue. (D) Tissue from a patient with squamous cell carcinoma of the larynx displays cytoplasmic and some nuclear staining. (E) Similarly, squamous cell carcinoma of the tongue immunolocalises activin β_{C} subunit protein in the cytoplasm with some focal nuclear staining. (F) Tissue from a patient with malignant stromal tumour of the small intestine displayed strong activin β_{C} subunit protein localisation. (G) In the normal small intestine, non-uniform activin β_{C} subunit localisation was observed in the epithelial cells.

FIG. 15 shows immunolocalisation of activin β_{C} subunit protein in patients with benign breast tissue, intraduct carcinoma and infiltrating lobular and ductal carcinoma. (A) Strong activin β_{C} subunit localisation is observed in the cytoplasm of cells within intraduct carcinoma (high grade). (B) Low level activin β_{C} subunit staining in the stromal cells in benign breast tissue is seen in the same patient. (C) Activin β_{C} subunit protein localises to the cytoplasm of the malignant cells, with nuclear staining observed intermittently, in infiltrating lobular carcinoma (BRE grade 2). (D) No localisation of the activin β_{C} subunit is observed in tumour cells in infiltrating ductal carcinoma (BRE grade 2).

FIG. 16 shows immunolocalisation of activin β_{C} subunit protein in patients with colon cancer. (A) Tissue from a patient with poorly differentiated colon adenocarcinoma strongly immunolocalises activin β_{C} subunit in the cytoplasm of tumour cells. (B) Adjacent regions of benign tissue in the same patient have weaker activin β_{C} subunit immunolocalisation in the colon surface epithelium and surrounding stroma. (C) A patient with moderately differentiated colon adenocarcinoma immunolocalised the activin β_{C} subunit in tumor cells intermittently, and adjacent benign cells displayed weak activin β_{C} subunit protein staining in the surface epithelium and some stroma (D). (E) The malignant regions of tissue from a patient with moderately well differentiated adenocarcinoma did not immunolocalise the activin β_{C} subunit in tumour cells. (F) Non-malignant regions of this tissue in this same patient displayed some weak activin β_{C} subunit localisation in stromal cells.

FIG. 17 shows immunolocalisation of activin β_{C} subunit protein in patients with gastric cancer. (A) In a patient with well differentiated gastric adenocarcinoma, activin β_{C} subunit protein was not detected in the tumor cells, however weak staining was observed in the stromal and epithelial cells in adjacent non-malignant regions (B). (C) Tumor cells from a patient with poorly differentiated gastric adenocarcinoma of intestinal type immunolocaised activin β_{C} subunit protein intermittently in the nuclei of tumor cells. (D) Tissue from a patient with signet ring gastric adenocarcinoma predominantly immunolocalised activin β_{C} subunit protein in nuclei of tumour cells, however some cytoplasmic staining was observed. (E) Tissue from a patient with poorly differentiated gastric adenocarcinoma with focal signet ring, arising at the gastro-oesophageal junction change had strong activin β_{C} subunit protein localized to the tumor cells, however adjacent normal oesophageal mucosa was negative for the activin β_{C} subunit (F).

FIG. 18 shows immunolocalisation of activin β_{C} subunit protein in brain tissue from patients with metastatic melanoma within brain.

(A) Metastatic melanoma cells show strong cytoplasmic immunolocalisation of the activin β_{C} subunit protein. (B) By contrast, another patient with melanoma metastastic to brain did not localize the activin β_{C} subunit protein in the tumour cells.

FIG. 19 shows immunolocalisation of activin β_{C} subunit protein in tissue from patients ho have primary tumours of the brain; meningioma and schwannoma. Meningioma are derived from meningiothelial cells. Schwanoma are of Schwann cell origin. (A) Strong immunolocalisation of activin β_{C} subunit protein in the cytoplasm of meningioma cells. In particular, this patient also displayed strong cell membrane immunolocalisation. (B) Schwanoma localizes the activin β_{C} subunit protein strongly in the cytoplasm of the tumour cells. A patient with higher grade (WHO grade 2) mengimoma had areas of both strong cytoplasmic staining (C) as well as areas of less intense cytoplasmic staining of activin β_{C} subunit protein (D) in these high grade regions.

FIG. 20 shows immunolocalisation of activin β_{C} subunit protein in tissue from patients with bladder cancer. (A) Tumor cells from a patient with transitional cell carcinoma (high grade) immunolocalises the activin β_{C} subunit protein weakly in the cytoplasm. (B) Tumor cells, from another patient with high grade transitional cell carcinoma, immunolocalise activin β_{C} subunit protein intermittently in both the cytoplasm and nuclei.

FIG. 21 shows immunolocalisation of activin β_{C} subunit protein in tissue from patients with thyroid cancer. (A) In the benign thyroid follicles from a patient with medullary cancer, activin β_{C} subunit protein was localized intermittently in the cytoplasm of the follicular epithelium. (B) In the adjacent tumor cells from the same patient with medullary cancer, strong cytoplasmic staining in the tumour cells was observed. (C) In a patient with follicular carcinoma of the thyroid, tumour cells immunolocalised the activin β_{C} subunit in the cytoplasm. (D) Papillary carcinoma of the thyroid displayed focal staining in the tumor cells, with some but not all cells immunolocalising activin β_{C} subunit protein.

FIG. 22 shows the expression of activin β_{C} subunit protein dimers in the using Western blot analysis. Proteins were separated by 15% SDS-PAGE gel. Lane 1: normal human patient A, 1.67 ul serum. Lane 2: normal patient A, 3.33 ul serum. Lane 3: normal human patient B, 3.33 ul serum. In lanes 1, 2 and 3 a strong, approximately 20 kDa band (arrow), was detected indicating the presence of either activin C (β_{C}-β_{C}) or activin BC (β_{B}-β_{C}) which have approximate sizes of 20 and 21 kDa, respectively. Lane 3 also had a faint band approximately 23 kDa (arrow) in size, which may be indicative of the presence of activin AC (β_{A}-β_{C}) that has an approximate size of 23 kDa.

### DETAILED DESCRIPTION OF THE INVENTION

The present invention is predicated, in part on the surprising determination that changes in the level of activin β_{C} subunit, relative to normal levels and whether in dimeric or monomeric form, is indicative of the onset of a disease condition, in particular a neoplastic condition. More specifically, it has been determined that activin β_{C} levels and bioactivity in many tissues show changes correlating to onset or establishment of a disease or condition. It serves as an indicator of change in tissues and cell function and characteristics of the cell. Accordingly, this correlation has facilitated the development of a simple yet sensitive test for neoplasia, in particular, this test being useful to diagnose, predict or monitor a neoplastic condition. Also facilitated has been the national design of therapeutic and prophylactic means of treating conditions characterised by aberrant, in particular uncontrolled, cell growth.

Accordingly, one aspect of the present invention is directed to a method of detecting in a mammal the onset, or predisposition to the onset, of a condition characterised by modulation of the level or bioactivity of activin β_{C}, which level is modulated relative to normal levels, said method comprising screening for the level of activin β_{C} protein and/or gene expression in a biological sample derived from said mammal.

Reference to “activin Pc” should be understood as a reference to all forms of activin β_{C} and to fragments, derivatives, mutants or variants thereof. “Activin β_{C}” is also interchangeable referred to as “activin β_{C} subunit”. It should also be understood to include reference to any isoforms which may arise from alternative splicing of activin β_{C} mRNA or mutant or polymorphic forms of activin β_{C}. Reference to “activin β_{C}” is not intended to be limiting and should be read as including reference to all forms of activin β_{C} including any protein encoded by the activin β_{C} subunit gene, any subunit polypeptide such as precursor forms which may be generated, and any activin β_{C} protein, whether existing as a monomer, multimer or fusion protein. Multimeric protein forms of activin β_{C} include for example the homodimeric activin C (β_{C}-β_{C}) or the heterodimeric activin AC (β_{A}-β_{A}), activin BC (β_{B}-β_{C}), activin CD (β_{C}-β_{D}) or activin CE (β_{C}-β_{E}) proteins. Accordingly, it should be understood that one may screen for activin β_{C} in its monomeric, homodimeric or heterodimeric form.

Without limiting the present invention to any one theory or mode of action, activin β_{C} has a structure similar to other activins and other members of the TGFβ superfamily. The structure of activins are based on the conservation of the number and spacing of the cysteines within each subunit and the disulphide linkages between the two subunits that form characteristic cysteine knots. Other similarities relate to dimer formation, the location of the bioactive peptide in the carboxy terminal region of the precursor activin subunit molecule and similar intracellular signalling mechanisms. Human activin β_{C}, in comparison with other TGF-β superfamily members, reveals a typical structure with 9 conserved cysteines and a large precursor molecule that contain a core of hydrophobic amino acids at the N terminus thought to be the secretion signal sequence (Hotten G et al, 1995, supra). The mouse activin β_{C} also contains 9 conserved cysteines and N terminal hydrophobic amino acids that may serve as a signal peptide (Schmitt et al. 1996, supra).

Without limiting the present invention to any one theory or mode of action, it has been determined that changes, that is, increases or descreases in the level of activin β_{C} relative to normal levels of activin β_{C} is indicative of the onset of a disease state. Accordingly, this determination has facilitated the development of diagnostic and prognostic techniques based on screening for the up- or down-regulation of activin β_{C} relative to normal levels.

More particularly, the present invention is directed to a method of detecting the onset, or predisposition to the onset, of a condition characterised by an increase in the level or bioactivity of activin β_{C} in a mammal, relative to normal levels, said method comprising screening for the level of activin β_{C} protein and/or gene expression in a biological sample derived from said mammal.

In another particular aspect, the present invention is directed to a method of detecting the onset, or predisposition to the onset, of a condition characterised by a decrease in the level or bioactivity of activin β_{C} in a mammal, relative to normal levels, said method comprising screening for the level of activin β_{C} protein and/or gene expression in a biological sample derived from said mammal.

Reference to a “condition characterised by modulation of the level or bioactivity of activin β_{C}” should be understood as a reference to any condition which is characterised by modulation, in particular an increase, in the level of activin β_{C} monomer or dimer. It should be understood that the change in the level of activing β_{C} may either be the cause or consequence of the onset of the disease condition.

Without limiting the present invention in any way, the disease or condition may include diseases or conditions of the pancreas, brain and neural tissue, adrenal gland, thyroid gland, stomach, colon, urinary bladder, endometrium, breast, lymph node, pancreas, brain and neural tissue, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum, oesophagus, myometrium and soft tissue.

In this regard, activin β_{C} subunit protein has been surprisingly found in human tissues of the following organs (normal, benign and malignant tumours): adrenal gland, thyroid gland, stomach, colon, rectum, urinary bladder, skin, breast, lymph node, human salivary gland, bone, nasal cavity, duodenum, gallbladder, uterine, cervix, pancreas, oesophagus, thyroid, thymus, brain, larynx, tongue, and small intestine.

Activin β_{C} subunit protein was also detected by applicants in human tissues of the following organs (both normal or disorders of): myometrium, uterus, fallopian tube, tonsil, seminal vesicle, spleen, soft tissue and appendix. Applicants have also detected activin AC and other dimers containing the β_{C} subunit protein in samples of normal human serum. The presence of β_{C} and the difference in patterns of the normal and tumor tissues indicates a change in cells and tissue characteristics indicative of disease or changes in the condition of the cells or tissue.

These results are surprising as the activin β_{C} subunit has frequently been referred to in the literature as a “liver specific activin”. Fang et al. 1997, Biochem Biophys Res Commun. 231(3):655-61 described activin β_{C} expression in adult mice as “a unique liver-restricted pattern” (Fang J et al, 1997, Biochem Biophys Res Commun. 231(3):655-61) Furthermore, Lau et al. stated that activin β_{C} exhibited a “highly restricted tissue expression pattern” in the mammalian liver (Lau et al, 2000. Mol Cell Biol. 20(16):6127-37) while Schmitt et al. found “that the inhibin/activin β_{C} gene is predominantly expressed in adult mouse liver” and that the expression level in liver was “specific and high” (Schmitt et al, 1996, Genomics 32:358-66) In addition, Chang et al. described activin β_{C} as being “expressed primarily in the liver in the adult” and having a “highly restricted tissue-specific expression pattern” (Chang et al, 2001, Mol Cell Endocrinol. Jun 30;180(1-2):39-46). Also Kron et al. stated that “the β_{C} subunit is exclusively expressed in liver tissue” (Kron et al, 1998, J Virol Methods 72:9-14)

Preferably, said condition is a neoplastic condition.

Even more preferably, said neoplastic condition is a malignant neoplasia of the pancreas, brain and neural tissue, adrenal gland, thyroid gland, stomach, colon, , urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum or oesophagus, myometrium and soft tissue or a non-malignant neoplasia of the fallopian tube, uterus, tonsil, spleen, appendix, seminal vesicle, myometrium and soft tissue.

Most preferably said activin β_{C} levels is either increased or decreased.

Accordingly, the present invention preferably provides a method of detecting the onset, or predisposition to the onset, of a neoplasm in a mammal said method comprising screening for the level or bioactivity of activin β_{C} protein and/or gene expression in a biological sample derived from said mammal wherein an increase in the level of activin β_{C} protein and/or gene expression relative to normal levels is indicative of the onset, or predisposition to the onset, of said neoplasm.

Reference to a “neoplasm” should be understood as a reference to an encapsulated or unencapsulated growth of neoplastic cells. Reference to a “neoplastic cell” should be understood as a reference to a cell exhibiting abnormal growth. The term “growth” should be understood in its broadest sense and includes reference to proliferation.

The phrase “abnormal growth” in this context is intended as a reference to cell growth which, relative to normal cell growth, exhibits one or more of an increase in the rate of cell division, an increase in the number of cell divisions, an increase in the length of the period of cell division, an increase in the frequency of periods of cell division or uncontrolled proliferation. Without limiting the present invention in any way, the common medical meaning of the term “neoplasia” refers to “new cell growth” that results as a loss of responsiveness to normal growth controls, eg. to neoplastic cell growth. A “hyperplasia” refers to cells undergoing an abnormally high rate of growth. However, as used herein, the terms “neoplasia” and “hyperplasia” can be used interchangeably, referring generally to cells experiencing abnormal cell growth rates. Neoplasias and hyperplasias include “tumours” which may be either benign, pre-malignant or malignant. The term “neoplasm” should be understood as a reference to a lesion, tumour or other encapsulated or unencapsulated mass or other form of growth which comprises neoplastic cells.

The term “neoplasm”, in the context of the present invention should be understood to include reference to all types of cancerous growths or oncogenic processes, metastatic tissues or malignantly transformed cells, tissues or organs irrespective of histopathologic type or state of invasiveness.

The neoplastic cells comprising the neoplasm may be any cell type, derived from any tissue, such as an epithelial or non-epithelial cell. Although the present invention is preferably directed to the diagnosis of malignant neoplasms, the diagnosis and/or monitoring of non-malignant neoplasms is not excluded. Reference to the terms “malignant neoplasm” and “cancer” herein should be understood as interchangeable. Preferably, said neoplasm is a neoplasm of the adrenal gland, thyroid gland, stomach, colon, rectum, urinary bladder, skin, breast, lymph node, human salivary gland, bone, nasal cavity, duodenum, gallbladder, uterine cervix, pancreas, oesophagus, thryoid, thymus, brain, larynx, tongue, and small intestine, myometrium, uterus, fallopian tube tonsil, seminal vesicle, spleen, soft tissue or appendix.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animals (eg. kangaroos, deer, foxes). Preferably, the mammal is a human or a laboratory test animal. Even more preferably, the mammal is a human.

Reference to a “biological sample” should be understood as a reference to any sample of cells or tissue which is derived from an organism. The cells may be single cells, cultured cells or part of a tissue. In this regard, the biological sample may be derivable from any human or non-human mammal, as detailed above. It should be further understood that reference to “organism” includes reference to embryos and foetuses.

The biological sample may be any sample of material derived from the organism. This includes reference to both samples which are naturally present in the organism, such as tissue and body fluids in a mammal (for example biopsy specimens such as lymphoid specimens, resected tissue, tissue extracts, blood, lymph fluid, faeces, bronchial secretions or cell culture medium) and samples which are introduced into the body of the organism and subsequently removed, such as, for example, the saline solution extracted from the lung following a lung lavage or from the colon following an enema. It also includes reference to cells which originated from an organism but have been maintained in vitro, for example cell lines, or which have been manipulated or treated subsequently to removal from the organism, for example immortalised or genetically modified cells or tissues.

The biological sample which is tested according to the method of the present invention may be tested directly or may require some form of treatment prior to testing. For example, a biopsy sample may require homogenisation prior to testing. Where the sample comprises cellular material, it may be necessary to extract or otherwise expose the nucleic acid material present in the cellular material in order to facilitate analysis of the nucleic acid material in terms of its mRNA expression, for example. In yet another example, the sample may be partially purified or otherwise enriched prior to analysis. For example, to the extent that a biological sample comprises a very diverse cell population, it may be desirable to select out a sub-population of particular interest.

The choice of what type of sample is most suitable for testing in accordance with the method disclosed herein will be dependent on the nature of the condition which is being monitored. For example, if the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one organism.

Although the method of the present invention is most conveniently performed by analysis of an isolated biological sample, it should also be understood that reference to analysing a sample “derived from” a mammal includes reference to analysing the sample in vivo.

The present invention is predicated on the finding that levels of activin β_{C} expression are modulated in neoplastic tissue as compared to normal tissue. In this regard, the person of skill in the art will understand that one may screen for changes to activin β_{C} at either the protein or the encoding nucleic acid molecule level. To the extent that it is not always specified, reference herein to screening for the level of “activin β_{C}” should be understood to include reference to screening for either the activin β_{C} protein or its encoding primary RNA transcript or mRNA. Accordingly, it should be understood that the present invention is directed to the correlation of the level of activin β_{C} relative to normal levels of this molecule. The “normal” level is the level of activin β_{C} protein or encoding nucleic acid molecule in a biological sample corresponding to the sample being analysed of an individual who has not developed the condition in issue nor is predisposed to developing said condition. The “normal” level also includes reference to the level of activin β_{C} in non-malignant regions of the tissue which is the subject of analysis. This latter method of analysis is a relative form of analysis in terms of the normal and test levels being determined from diseased and test tissues, respectively, derived from a single individual. However, the method of the present invention should also be understood to encompass non-relative analyses means such as the analysis of test results relative to a standard result which reflects individual or collective results obtained from healthy individuals, other than the patient in issue. Said “normal level” may be a discrete level or a range of levels. Individuals exhibiting activin β_{C} levels higher or lower than the normal range are generally regarded as having undergone the onset of the condition or may be predisposed to the onset of the condition. In this regard, it should be understood that activin β_{C} levels may be assessed or monitored by either quantitative or qualitative readouts. The reference level may also vary between individual forms (such as differently processed forms) of activin β_{C} molecules.

Accordingly, the terms “increase”, “decrease” and “modulation” refer to increases and decreases in activin β_{C} levels or bioactivity relative either to a normal reference level (or normal reference level range) or to an earlier result determined from the patient in issue, this latter reference point being particularly relevant in the context of the ongoing monitoring of a patient, as hereinafter described.

Although the preferred method is to detect a change in activin β_{C} levels in order to diagnose the onset of or predisposition to the onset of, in a preferred embodiment, a neoplasm, the detection of the converse change in activin β_{C} levels may be desired under certain circumstances. For example, where a surgical procedure such as a breast lumpectomy is performed rather than a mastectomy, one may seek to monitor for improvement in the disease state of the breast, and its prognostic implications in relation to the development of further tumours during the course of therapeutic treatment of the patient. Alternatively, patients presenting with symptoms of a neoplastic condition or a genetic or environmental predisposition to the development of a neoplastic condition may be monitored. In another example, to the extent that a biopsy or other test has been performed and analysis thereof has revealed a predisposition to the development of a neoplastic condition, one may seek to monitor systemic or appropriately selected localised levels of activin β_{C} as an indication of the development or regression of metastases. This aspect of the present invention therefore enables one to monitor the progression of a neoplastic condition or predisposition thereto. It should be understood that in accordance with this aspect of the present invention, activin β_{C} levels will likely be assessed relative to one or more previously obtained results, as hereinbefore described.

The method of the present invention is therefore useful as a one off test or as an on-going monitor of those individuals thought to be at risk of neoplasm development or as a monitor of the effectiveness of therapeutic or prophylactic treatment regimes directed to inhibiting or otherwise slowing neoplasm development. In these situations, mapping the modulation of activin β_{C} expression in any one or more classes of biological samples is a valuable indicator of the status of an individual or the effectiveness of a therapeutic or prophylactic regime which is currently in use. Accordingly, the method of the present invention should be understood to extend to monitoring for increases or decreases in activin β_{C} levels in an individual relative to their normal level (as hereinbefore defined) or relative to one or more earlier activin β_{C} levels determined from said individual.

Accordingly, another aspect of the present invention is directed to a method of monitoring for the onset or progression of a condition characterised by modulation of the level or bioactivity of activin β_{C} in a mammal, which level is modulated relative to normal levels, said method comprising screening for the level of activin β_{C} protein and/or gene expression in a biological sample derived from said mammal.

In one particular aspect, the present invention is directed to a method of monitoring for the onset or progression of a condition characterised by an increase in the or bioactivity of activin β_{C} in a mammal, relative to normal levels, said method comprising screening for the level of activin β_{C} protein and/or gene expression in a biological sample derived from said mammal.

In another aspect, the present invention is directed to a method of monitoring for the onset or progression of a condition characterised by a decrease in the or bioactivity of activin β_{C} in a mammal, relative to normal levels, said method comprising screening for the level of activin β_{C} protein and/or gene expression in a biological sample derived from said mammal.

Preferably, said condition is a malignant or non-malignant neoplasm.

Still more particularly, there is provided a method of monitoring for the onset or progression of a neoplasm in a mammal said method comprising screening for the level or bioactivity of activin β_{C} protein and/or gene expression in a biological sample derived from said mammal wherein an increase in the level of activin β_{C} protein and/or gene expression relative to the normal level of activin β_{C} is indicative of the onset or progression of said neoplasm.

Preferably, said neoplastic condition is a malignant neoplasia of the pancreas, brain and neural tissue, adrenal gland, thyroid gland, stomach, colon,, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum or oesophagus, myometrium and soft tissue or a non-malignant neoplasia of the fallopian tube, uterus, tonsil, spleen, appendix, seminal vesicle, myometrium and soft tissue.

The method of the present invention has widespread applications including, but not limited to, the diagnostic or prognostic analysis of neoplasms or any condition characterised by a change, from normal levels, in activin β_{C} levels.

Means of screening for changes in activin β_{C} levels in an individual, or biological sample derived therefrom, can be achieved by any suitable method, which would be well known to the person of skill in the art, such as but not limited to:
(i) In vivo detection of activin β_{C}. Molecular Imaging may be used following administration of imaging probes or reagents capable of disclosing altered expression levels of the activin β_{C} mRNA or protein expression product in the prostate tissues.
   Molecular imaging (Moore, A., Basilion, J., Chiocca, E., and Weissleder, R., BBA, 1402:239-249, 1988; Weissleder, R., Moore, A., Ph.D., Mahmood-Bhorade, U., Benveniste, H., Chiocca, E. A., Basilion, J. P. Nature Medicine, 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy. Historically, detection of malignant tumor cells in a background of normal or hyperplastic benign tissue is often based on differences in physical properties between tissues, which are frequently minimal, resulting in low contrast resolution. Application of expression profiling will define the differences in “molecular properties” between cancer and normal tissues that arise as a result of malignant transformation.
(ii) Detection of up-regulation of mRNA expression in the cells by Fluorescent In Situ Hybridization (FISH), or in extracts from the cells by technologies such as Quantitative Reverse Transcriptase Polymerase Chain Reaction (QRTPCR) or Flow cytometric qualification of competitive RT-PCR products (Wedemeyer, N., Potter, T., Wetzlich, S. and Gohde, W. Clinical Chemistry 48:9 1398-1405, 2002) or array technologies.
   For example, a labelled polynucleotide encoding activin β_{C} may be utilized as a probe in a Northern blot of an RNA extract obtained from the prostate. Preferably, a nucleic acid extract from the animal is utilized in concert with oligonucleotide primers corresponding to sense and antisense sequences of a polynucleotide encoding activin β_{C}, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™) are used for the detection of nucleic acids as, for example, described by Fodor et al., 1991 and Kazal et al., 1996. The above genetic techniques are well known to persons skilled in the art.

For example, to detect activin β_{C} encoding RNA transcripts, RNA is isolated from a cellular sample suspected of containing activin β_{C} RNA, e.g. total RNA isolated from human prostate cancer tissue. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.
“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987; Erlich, 1989. Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian activin β_{C} genes. For example, one primer is prepared which is predicted to anneal to the antisense strand and another primer prepared which is predicted to anneal to the sense strand of a cDNA molecule which encodes activin β_{C}.
To detect the amplified product, the reaction mixture is typically subjected to agarose gel electrophoresis or other convenient separation technique and the relative presence of the activin β_{C} specific amplified DNA detected. For example, activin β_{C} in amplified DNA may be detected using Southern hybridization with a specific oligonucleotide probe or comparing is electrophoretic mobility with DNA standards of known molecular weight. Isolation, purification and characterization of the amplified activin β_{C} DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of activin β_{C}. The relative amounts of activin β_{C} mRNA and cDNA can then be determined.
(iii) Measurement of altered activin β_{C} protein levels in cell extracts or blood or other suitable biological sample, either qualitatively or quantitatively, for example by immunoassay, utilising immunointeractive molecules such as antibodies to detect β_{C} hetero- or homodimers or β_{C} monomeric subunits.
   In one example, one may seek to detect activin β_{C}-immunointeractive molecule complex formation. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.
   Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen. The antigen in this case is activin β_{C} or a fragment thereof.
   Two-site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent.
   In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.
   An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:-
(a) direct attachment of the reporter molecule to the antibody;
(b) indirect attachment of the reporter molecule to the antibody; i.e., attachment of the reporter molecule to another assay reagent which subsequently binds to the antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Nos. 4,366,241, 4,843,000, and 4,849,338. Suitable enzymes useful in the present invention include alkaline phosphatase, horseradish peroxidase, luciferase, p-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. Nos. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.
(iv) The use of aptamers in screening for nucleic acid molecules or expression products
(v) Determining altered protein expression based on any suitable functional test, enzymatic test or immunological test in addition to those detailed in point (iii), above.

It should be understood that in addition to screening for the levels of activin β_{C} subunit protein or mRNA expression levels, it may also be desirable to determine whether the subject β_{C} subunit exists in monomeric or dimeric form and if dimeric, then the nature of the dimer which it has formed. Accordingly, the present invention extends to introducing additional steps to the screening method which is utilised, which additional steps are directed to screening for one or more of the activin β_{A}, β_{B}, β_{D} or β_{E} subunits. This analysis may be performed simultaneously or sequentially with the analysis performed in relation to β_{C}. Further, the analysis in relation to the presence or otherwise of one of these non β_{C} subunits can be performed utilising the techniques hereinbefore described.

Bioactivity of the activin β_{C} subunit may be determined by the ability of the activin β_{C} subunit to induce or decrease activin dimer formation. The dimer formation may be measured by the presence of the activin p subunit by using an antibody to the subunit.

As detailed above, any suitable technique may be utilised to detect activin β_{C} or its encoding nucleic acid molecule. The nature of the technique which is selected for use will largely determine the type of biological sample which is required for analysis. Such determinations are well within the scope of the person of skill in the art.

In a particularly preferred embodiment, the detection assay of the present invention is performed utilising an antibody based method.

In one example, said detection may be directed to activin β subunit dimer molecules.

Accordingly, there is provided a method of detecting the onset, or predisposition to the onset, of a condition characterised by modulation of the level of activin β_{C} in a mammal, which level is modulated relative to normal levels, said method comprising:
(a) contacting a first antibody that recognises an epitope of a first activin β subunit with a biological sample derived from said mammal;
(b) allowing the first antibody to bind to said first activin β subunit in said sample;
(c) washing said sample to substantially remove unbound material;
(d) contacting said sample with a second antibody that recognises an epitope of a second activin β subunit, wherein the second antibody is tagged with a labelling agent; and
(e) detecting the labelling agent to identify an activin β_{C} subunit dimer in said sample, wherein the first or second antibody recognises an epitope of an activin β_{C} subunit.

Preferably, said condition is a neoplastic condition and said modulation is an increase in the level of activin β_{C} subunit.

Preferably, the activin β_{C} dimer detected is selected from the group consisting of activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer to be detected is activin AC(β_{A}-β_{C}). In the method it is preferred that the first antibody recognises an epitope of an activin β_{C} subunit. Preferably, the second antibody recognises an epitope of an activin β_{A} or β_{B} subunit. More preferably, the second antibody recognises an epitope of an activin β_{A} subunit. Preferably, step (e) includes quantifying the amount of an activin β_{C} dimer in the cell or biological sample. The steps of the method may be performed as previously described for detecting an activin β_{C} subunit.

It should also be understood, however, that this is not a limitation and that the present invention extends to detecting the activin β_{C} subunit, alone, irrespectively of whether it is in monomeric or dimeric form.

In the diagnostic methods of the present invention it is preferred that the subject is a mammalian animal, including but not limited to a human. The biological sample of the subject is preferably serum, tissue culture supernatant, seminal plasma, cell lysates, tissue homogenates, biological fluids, cerebrospinal fluid, or seminal fluid. The biological sample may be a lysate of tissue or conditioned media of cells, particularly if the disease or condition to be diagnosed is cellular. If the disease or condition to be diagnosed is related to a reproductive disease or condition then the biological sample may include ovarian follicular fluid, seminal fluid or seminal plasma.

The term “antibody” as used in accordance with this preferred embodiment is used in the broadest sense and specifically covers monoclonal antibodies, polyclonal antibodies, multispecific antibodies (e.g., bispecific antibodies), and antibody fragments so long as they bind specifically to a target antigen. Antibodies may be obtained from commercial sources.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a ingle determinant on the antigen. The modifier “monoclonal” indicates the character of he antibody as being obtained from a substantially homogeneous population of antibodies, and is not to be construed as requiring production of the antibody by any particular method. For example, the monoclonal antibodies to be used in accordance with the present invention may be made by the hybridoma method, isolated from phage antibody libraries, or may be made by recombinant DNA methods. The monoclonal antibodies may also be obtained from commercial sources.

Therefore, suitable antibodies specific to activin β_{C} can include, but are not limited to, polyclonal, monoclonal, chimeric, single chain, Fab fragments, and a Fab expression library. For preparation of monoclonal antibodies directed towards activin β_{C} protein, any technique that provides for the production of antibody molecules by continuous cell lines in culture may be used. Such techniques include, but are not restricted to, the hybridoma technique originally developed by Kohler and Milstein (Kohler G, Milstein C 1975, Nature 256:495-7), the trioma technique, the human B-cell hybridoma technique (Kozbar 1983 Immunology Today 4:72), and the EBV hybridoma technique to produce human monoclonal antibodies (Cole 1985 Monoclonal Antibodies and Cancer Therapy. In. Alan R. Liss, Inc: 77-96).

Various procedures known in the art may be used for the production of polyclonal antibodies to an activin β_{C} protein. For production of the antibody, various host animals can be immunized by injection with activin β_{C} protein, such host animals include, but are not limited to, rabbits, mice, rats, etc. Various adjuvants can be used to increase the immunological response, depending on the host species, and include, but are not limited to, Freud's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Calmette-Guerin (BCG) and corynebacterium parvum.

Suitable antibodies that specifically bind to activin β_{C} can be introduced into a cell in numerous fashions, including, for example, microinjection of antibodies into a cell Morgan and Roth, 1988, Immunol Today 9:84-8) or transforming hybridome mRNA encoding a desired antibody into a cell (Burke and Warren, 1984, Cell 36:847-56).

Antibody fragments can be generated by techniques known in the art. For example, such fragments include, but are not limited to, the F(ab′)₂ fragment which can be produced by pepsin digestion of the antibody molecule; the Fab′ fragments that can be generated by reducing the disulphide bridges of the F(ab′)₂ fragment, the Fab fragments that can be generated by treating the antibody molecule with papain and a reducing agent, and Fv fragments.

In a further technique, recombinant antibodies specific to activin β_{C} protein can be engineered and ectopically expressed in a wide variety of cell types to bind to activin β_{C} as well as to block activin β_{C} from dimerising.

The preparation and use of antibodies according to the present invention may be achieved using techniques well known in the art, and include various antibody labelling techniques and applications. Suitable labels for antibodies include, but are not limited to, radionucleotides, enzymes, substrates, cofactors, inhibitors, fluorescent agents, chemiluminescent agents, magnetic particles and the like. The antibody may also be treated prior to adding the label, for example by biotinylation.

The term “label” when used herein refers to a compound or composition which is conjugated or fused directly or indirectly to a reagent such as an antibody and facilitates detection of the reagent to which it is conjugated or fused. The label itself may be detectable (e.g., radioisotope labels or fluorescent labels) or, in the case of an enzymatic label, may catalyze chemical alteration of a substrate compound or composition which is detectable. Labelling may include the addition of a subsequent step with a label for example, biotin step, then strepavidin-alkaline phosphatase label.

Labelling of the antibody may be achieved directly or indirectly. Well known conjugation methods may be used for attaching labels to antibodies. Preferably, after labelling, unbound label is removed from the labelled antibody using purification procedures known to those of skill in the art. The antibody may also be fractionated to provide an immunoglobulin fraction such as IgG or IgM fractions. These antibody fractions may be isolated using methods known to those in the art including using recombinant protein G for IgG or immunoprecipitation for IgM.

It is most preferred that the activin β_{C} monomers or dimers including β_{C} are detected by an antibody, wherein the antibody recognises an epitope of an activin β_{C} subunit. Preferably, the antibody is capable of recognising monomeric or dimeric forms of activin β_{C}. More preferably, the antibody recognises an epitope of activin β_{C} said epitope comprising the amino acid sequence VPTARRPLSLLYYDRDSNIVKTDIPDMVVEAC (SEQ ID NO:1) or an equivalent thereof. It is preferred that the antibody is a monoclonal antibody. Preferably, the antibody is specific to an activin β_{C} subunit. More preferably, the antibody is specific to the human activin β_{C} subunit. The antibody may be a mouse monoclonal antibody developed against the human activin β_{C} subunit. Most preferably, the antibody does not cross react with activin β_{A}, β_{B} or β_{E} peptides.

Preferably, the activin β_{C} antibody is used in an ELISA based method for the diagnostic method of the present invention.

In a most preferred embodiment, the present invention is directed to detecting levels of activin AC (activin β_{A}-β_{C} dimer). In the context of this preferred aspect of the present invention, and in terms of the preferred antibody based methods utilised herein, the selected antibody based screening assay utilises a first antibody directed to an epitope of an activin β_{C} subunit and a second antibody directed to an epitope of an activin β_{A} subunit.

In a most preferred embodiment, the diagnostic methods described herein include the step of quantifying the amount of activin β_{C} monomer or dimer present in the biological sample which is the subject of testing.

The biological sample may be pretreated before contacting the sample with the first antibody. For instance, the sample may be diluted with a suitable diluent, such tissue culture media and/or PBS. The sample may preferably be denatured with SDS by heating before contacting the sample with the first antibody. The biological sample may preferably be treated to oxidise the sample. More preferably, activin β_{C} subunit in the sample is oxidised, such that a methionine on an activin β_{C} subunit is oxidised. A suitable oxidising agent, such as H₂O₂ may be added to the biological sample to oxidise the methionine on an activin β_{A} subunit.

In a preferred embodiment, the method includes the additional step of adding a dissociating agent to the sample to remove binding proteins. Preferably, the dissociating agent is added before step (a). Preferably, the binding protein removed is selected from the group consisting of follistatins, BMPs or α-2 macroglobulins. SDS may preferably be added to sample as a dissociating agent to remove binding proteins such as follistatins, BMPs, α-2 macroglobulins and others). However other dissociating agents include those published in McFarlane et al, 1996 Eur J Endocrinol 134:481-9 which describes sodium deoxycholate, Tween 20, SDS as useful dissociating agents. Binding proteins such as follistatin bind to the β subunits of activin A, B with high affinity, and inhibin A and B with lower affinity. Follistatin may also bind to the activin β_{C} subunit. Therefore, it is preferable to include the dissociating step to remove binding proteins.

In step (b) of the method the first antibody is allowed to bind to a first activin β subunit in the sample. This is preferably achieved by incubating the first antibody and the biological sample under suitable conditions. For instance, suitable media including BSA and/or PBS may be used, preferably activin free serum is used. Most preferably, the sample is incubated over night in a humidified environment.

In step (c) of the method the sample is washed to substantially remove any unbound material in the sample. The sample is washed in any suitable washing solution, preferably including water or PBS. The sample is preferably washed such that the labelled antibody specifically binds to the target activin subunit.

In step (d) of the method the sample is contacted with a second antibody that recognises an epitope of a second activin β subunit. Preferably, the second antibody recognises an epitope of an activin β_{A}, β_{B}, β_{C}, β_{D} or β_{E} subunit. More preferably, the second antibody recognises an epitope of an activin β_{A} subunit. The second antibody may be a monoclonal or polyclonal antibody and may be generated by methods previously discussed.

The second antibody is required to be tagged with a labelling agent. The preparation and use of antibodies according to the present invention may be achieved using techniques well known in the art, and include various antibody labelling techniques and applications. Suitable labels for antibodies include, but are not limited to, radionucleotides, enzymes, substrates, cofactors, inhibitors, fluorescent agents, chemiluminescent agents, magnetic particles and the like. The antibody may also be treated prior to adding the label, for example by biotinylation.

The second antibody that is tagged by a labelling agent as hereinbefore described is typically referred to as the “tag antibody” and is preferably used in a colour detection method. The second antibody may be bound to a labelling agent, such as biotin wherein detection of the label is measured by a coloured enzyme reaction product. Other labelling preferably includes using activin p subunit antibody directly labelled with alkaline phosphatase.

In step (e) of the method an activin dimer that is bound to the second labelled antibody is detected. The method of detection would depend on the labelling agent used to tag the second antibody and then addition of strepavidin alkaline phosphatase. The detection preferably involves colour detection from kit reagents. For instance, colour may be read using a microplate reader using a standard. Calculations on levels or bioactivity of activin AC are based on a standard curve of known amounts of activin AC. For instance, bovine follicular fluid and a human recombinant or purified activin AC protein may be used as a standard for the activin AC assay. Preferably, step (e) includes quantifying the amount of an activin β_{C} dimer in the biological sample.

In an alternative embodiment, the method may be performed in the reverse manner (swapping the capture and tag antibodies). For example, an activin β_{A} antibody may be coated on the plate and an activin β_{C} antibody may be labelled. However, this is less preferable due to the high amounts of activin A (β_{A}-β_{A}) in certain samples which would cause decreased sensitivity of the assay.

Yet another aspect of the present invention is directed to a composition when used to detect the onset, or predisposition to the onset, of a condition characterised by modulation of the level or bioactiviy of activin β_{C}, in accordance with the methods hereinbefore described, said composition comprising an activin β_{C} detection means.

More preferably, the present invention provides a composition when used to detect the onset, or predisposition to the onset, of a condition characterised by modulation of the level or bioactiviy of activin β_{C}, wherein said composition comprises an antibody directed to an epitope of an activin β_{C} subunit together with a suitable diluent, excipient or carrier.

Preferably, said condition is a neoplastic condition.

Still more preferably, said antibody is capable of recognising the activin β_{C} subunit in the context of either its monomeric or dimeric formation. Most preferably, said antibody is directed to an epitope of activin β_{C} which comprises the amino acid sequence:

| | |
|---|---|
| VPTARRPLSLLYYDRDSNIVKTDIPDMVVEAC. | (SEQ ID NO:1) |

The compositions as herein before described preferably include a suitable diluent, excipient or carrier which is compatible with the activin β_{C} subunit detection means, in particular the subject antibody. An acceptable carrier, excipient or diluent may include, water, salt solutions, BSA, Triton X-100. Preferably, the compositions are sterile aqueous solutions. The compositions may also contain buffers, diluents and other suitable additives. The compositions may include other adjunct components that are compatible with the antibody that recognises an epitope of an activin β_{C} subunit, such as labelling agents or dyes.

Yet another aspect of the present invention provides a diagnostic kit for use in detecting the onset, or predisposition to the onset, of a condition characterised by modulation of the level or bioactivity of activin β_{C} subunit, said kit comprising an activin β_{C} subunit protein and/or encoding nucleic acid detection means in a first compartment and reagents useful for facilitating detection by said detection means in a second compartment. Further compartments may also be included, for example, to include means for facilitating the collection and storage of a biological sample.

Preferably, said detection means are antibodies as hereinbefore described.

In the kits of the present invention their may be included a first antibody and a second antibody as previously described. The first antibody preferably recognises an epitope of an activin β_{C} subunit. The second antibody recognises an epitope of an activin β_{A}, β_{B}, β_{C}, β_{D} or β_{E} subunit. More preferably, the second antibody recognises an epitope of an activin PA subunit. Preferably, the first or second antibody is a purified antibody is that is capable of recognising activin β_{C} in both its monomeric and dimeric forms. Still more preferably, the antibody is directed to an epitope of activin β_{C} comprising the amino acid sequence:

| | |
|---|---|
| VPTARRPLSLLYYDRDSNIVKTDIPDMVVEAC. | (SEQ ID NO:1) |

Without limiting the present invention to any one theory or mode of action, it is thought that the activin β_{C} subunit can dimerise with non-β_{C} activin subunits to inhibit formation of activin A, activin B, activin AB or other activin dimers. Accordingly, modulation of the levels or bioactivity of activin β_{C} provides a means of modulating the biological activity of these activin dimers. In a related aspect, therefore, the findings of the present invention now facilitate the rational design of methodology directed to the therapeutic and/or prophylactic treatment of the conditions hereinbefore described, in particular neoplastic conditions. These methods are based on the notion that down-regulating activin β_{C} levels down-regulates the formation of activin β_{C} comprising dimers thereby up-regulating levels of non- activin β_{C} subunit dimers, such as activin A, activin B or activin AB dimers. Whether or not up-regulation of non-activin β_{C} dimer formation is beneficial will depend on the nature of the condition in issue. As hereinbefore described, where an increase in activin β_{C} levels is indicative of the onset of neoplasia, one would seek to decrease activin β_{C} levels in the context of a therapeutic treatment regime in order to normalise levels of non-β_{C} activin dimers. The converse approach would be applicable where a decrease in activin β_{C} levels is associated with the onset of the condition in issue. Preferably, said condition is abnormal cell growth.

Accordingly, another aspect of the present invention is directed to a method of modulating the abnormal growth of a cell, said method comprising modulating the level or bioactivity of activin β_{C} subunit.

Preferably, said activin β_{C} subunit levels or bioactivity are modulated intracellularly.

It should be understood that the modulated levels of activin β_{C} subunit may be achieved by modulating either the monomeric or dimeric activin β_{C} subunit levels.

In one preferred embodiment, the present invention is directed to a method of modulating the abnormal growth of a cell, said method comprising modulating the level or bioactivity of activin β_{C} subunit wherein up-regulating activin β_{C} subunit levels or bioactivity to a functionally effective level induces said abnormal growth and down-regulating activin β_{C} subunit levels or bioactivity to a functionally ineffective level inhibits said abnormal growth.

In another preferred embodiment, the present invention is directed to a method of modulating the abnormal growth of a cell, said method comprising modulating the level or bioactivity of activin β_{C} subunit wherein down-regulating activin β_{C} subunit levels or bioactivity to a functionally ineffective level induces said abnormal growth and up-regulating activin β_{C} subunit levels or bioactivity to a functionally effective level inhibits said abnormal growth.

Even more particularly, the present invention is directed to a method of modulating the growth of a neoplastic cell, said method comprising modulating the level or bioactivity of activin β_{C} subunit wherein up-regulating activin β_{C} subunit levels or bioactivity to a functionally effective level induces said neoplastic growth and down-regulating activin β_{C} subunit levels or bioactivity to a functionally ineffective level inhibits said neoplastic growth.

Reference to “abnormal growth” and “neoplastic cell” should be understood to have the same meaning as hereinbefore provided.

Preferably, said neoplastic condition is a malignant neoplasia of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum or oesophagus, myometrium and soft tissue or a non-malignant neoplasia of the fallopian tube, uterus, tonsil, spleen, appendix, seminal vesicle, myometrium and soft tissue.

Reference to “activin β_{C} should be understood as having the same meaning as hereinbefore defined but additionally includes reference to homologues thereof. Preferably, said activin β_{C} is the activin β_{C} subunit as hereinbefore defined.

Reference to “modulating” should be understood as a reference to up-regulating or down-regulating the subject cell growth. Reference to “down-regulating” cell growth should therefore be understood as a reference to preventing, reducing (eg. slowing) or otherwise inhibiting one or more aspects of cell growth while reference to “up-regulating” should be understood to have the converse meaning. Although the preferred method is to down-regulate the growth of a neoplastic cell or to prevent the shift to a neoplastic state of a cell predisposed in this regard, the present invention nonetheless extends to up-regulating cell growth, which may be desired in certain circumstances. For example, one may seek to utilise the method of the present invention to induce a neoplastic cellular phenotype in vitro in order to enable one to screen for other potential therapeutic means.

In accordance with a preferred aspect, the present invention therefore preferably provides a method of down-regulating the growth of a neoplastic cell, said method comprising down-regulating the level or bioactivity of activin β_{C} subunit to a functionally ineffective level. Reference herein to attaining either a “functionally effective level” or “functionally ineffective level” of activin β_{C} subunit should be understood as a reference to attaining that level of activin β_{C} subunit at which modulation of cell growth can be achieved, whether that be up-regulation or down-regulation. In this regard, it is within the skill of the person of skill in the art to determine, utilising routine procedures, the threshold level of activin β_{C} subunit expression above which or below which cell growth is modulated. In accordance with the disclosures provided herein, these levels are preferably assessed relative to normal levels.

It should be understood that reference to an “effective level” means the level necessary to at least partly attain the desired response. The amount may vary depending on the health and physical condition of the cellular population and/or individual being treated, the taxonomic group of the cellular population and/or individual being treated, the degree of up or down-regulation which is desired, the formulation of the composition which is utilised, the assessment of the medical situation and other relevant factors. Accordingly, it is expected that this level may vary between individual situations, thereby falling in a broad range, which can be determined through routine trials.

Modulating activin β_{C} subunit levels or bioactivity may be achieved by any suitable means including, but not limited to:
(i) Modulating absolute levels of activin β_{C} subunit such that either more or less activin β_{C} subunit is present in the cellular environment.
(ii) Agonising or antagonising activin β_{C} subunit protein functional activity such that the functional effectiveness of activin β_{C} subunit is either increased or decreased. For example, increasing the half life of activin β_{C} subunit may achieve an increase in the functionally effective level of activin β_{C} subunit without actually necessitating an increase in the absolute concentration of activin β_{C} subunit. Similarly, the partial antagonism of activin β_{C} subunit may act to reduce, although not necessarily eliminate, the functional effectiveness of said activin β_{C} subunit.
   Accordingly, this may provide a means of down-regulating activin β_{C} subunit functioning without necessarily down-regulating absolute concentrations of activin β_{C} subunit.

In terms of achieving the up or down-regulation of activin β_{C} subunit, means for achieving this objective would be well known to the person of skill in the art and include, but are not limited to:
(i) Introducing into a cell a nucleic acid molecule encoding activin β_{C} subunit or in order to up-regulate the capacity of said cell to express activin β_{C} subunit.
(ii) Introducing into a cell a proteinaceous or non-proteinaceous molecule which modulates transcriptional and/or translational regulation of a gene, wherein this gene may be the activin β_{C} subunit gene or functional portion thereof or some other gene or gene region (eg. promoter region) which directly or indirectly modulates the expression of the activin β_{C} subunit gene.
(iii) Introducing into a cell the activin β_{C} subunit expression product.
(iv) Introducing a proteinaceous or non-proteinaceous molecule which functions as an antagonist to the activin β_{C} subunit expression product.
(v) Introducing a proteinaceous or non-proteinaceous molecule which functions as an agonist of the activin β_{C} subunit expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the activin β_{C} subunit expression product or small molecules capable of acting as agonists or antagonists. Chemical agonists may not necessarily be derived from the activin β_{C} subunit expression product but may share certain conformational similarities. Alternatively, chemical agonists may be specifically designed to meet certain physiochemical properties. Antagonists may be any compound capable of blocking, inhibiting or otherwise preventing activin β_{C} subunit from carrying out its normal biological function. Antagonists include monoclonal antibodies and antisense nucleic acids which prevent transcription or translation of activin β_{C} subunit genes or mRNA in mammalian cells. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) of activin β_{C} may be created or identified by their ability to suppress the expression of activin β_{C}. The production of antisense oligonucleotides for a given protein is described in, for example, Stein and Cohen, 1988 (Cancer Res 48:2659-68) and van der Krol et al., 1988 (Biotechniques 6:958-976).

The proteinaceous and non-proteinaceous molecules referred to in points (i)-(v), above, are herein collectively referred to as “modulatory agents”. Preferably, said modulatory agents are antibodies, to the extent that it is sought to decrease activin β_{C} subunit levels or bioactivity.

Screening for the modulatory agents hereinbefore defined can be achieved by any one of several suitable methods including, but in no way limited to, contacting a cell comprising the activin β_{C} subunit gene or functional equivalent or derivative thereof with an agent and screening for the modulation of activin β_{C} subunit protein production or functional activity, modulation of the expression of a nucleic acid molecule encoding activin β_{C} subunit or modulation of the activity or expression of a downstream activin β_{C} subunit cellular target. Detecting such modulation can be achieved utilising techniques such as Western blotting, electrophoretic mobility shift assays and/or the readout of reporters of activin β_{C} subunit activity such as luciferases, CAT and the like.

It should be understood that the activin β_{C} subunit gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate activin β_{C} subunit activity, at either the nucleic acid or expression product levels, or the gene may require activation—thereby providing a model useful for, inter alia, screening for agents which up-regulate activin β_{C} subunit expression. Further, to the extent that an activin β_{C} subunit nucleic acid molecule is transfected into a cell, that molecule may comprise the entire activin β_{C} subunit gene or it may merely comprise a portion of the gene such as the portion which regulates expression of the activin β_{C} subunit product. For example, the activin β_{C} subunit promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. In another example, the subject of detection could be a downstream activin β_{C} subunit regulatory target, rather than activin β_{C} subunit itself. Yet another example includes activin β_{C} subunit binding sites ligated to a minimal reporter. Modulation of activin β_{C} subunit activity can be detected by screening for the modulation of cellular growth. This is an example of an indirect system where modulation of activin β_{C} subunit expression, per se, is not the subject of detection. Rather, modulation of the down-stream activity which activin β_{C} subunit regulates is monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the activin β_{C} subunit nucleic acid molecule or expression product itself or which modulate the expression of an upstream molecule, which upstream molecule subsequently modulates activin β_{C} subunit expression or expression product activity. Accordingly, these methods provide a mechanism of detecting agents which either directly or indirectly modulate activin β_{C} subunit expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of activin β_{C} subunit or the activity of the activin β_{C} subunit expression product. Said molecule acts directly if it associates with the activin β_{C} subunit nucleic acid molecule or expression product to modulate expression or activity, respectively. Said molecule acts indirectly if it associates with a molecule other than the activin β_{C} subunit nucleic acid molecule or expression product which other molecule either directly or indirectly modulates the expression or activity of the activin β_{C} subunit nucleic acid molecule or expression product, respectively. Accordingly, the method of the present invention encompasses the regulation of activin β_{C} subunit nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example activin β_{C} subunit or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, activin β_{C} subunit or derivative thereof may be fused to a molecule to facilitate its entry into a cell. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” of activin β_{C} subunit should be understood to mean molecules which exhibit at least some of the functional activity of the form of activin β_{C} subunit of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of activin β_{C} subunit which exhibits similar and suitable functional characteristics to that of the activin β_{C} subunit which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic 25 molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing activin β_{C} subunit analogues which exhibit properties such as more potent pharmacological effects. Activin β_{C} subunit or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed. With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of activin β_{C} subunit one may identify and utilise molecules which function agonistically or antagonistically to activin β_{C} subunit in order to up or down-regulate the functional activity of activin β_{C} subunit in relation to modulating cellular growth. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of activin β_{C} subunit capable of acting as agonists or antagonists. Chemical agonists may not necessarily be derived from activin β_{C} subunit but may share certain conformational similarities. Alternatively, chemical agonists may be specifically designed to mimic certain physiochemical properties of activin β_{C} subunit. Antagonists may be any compound capable of blocking, inhibiting or otherwise preventing activin β_{C} subunit from carrying out its normal biological functions. Antagonists include monoclonal antibodies specific for activin β_{C} subunit or parts of activin β_{C} subunit.

Analogues of activin β_{C} subunit or of activin β_{C} subunit agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional amino acid | Code |
|---|---|
| α-aminobutyric acid | Abu |
| α-amino-α-methylbutyrate | Mgabu |
| aminocyclopropanecarboxylate | Cpro |
| aminoisobutyric acid | Aib |
| aminonorbornylcarboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carbamylmethyl)glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stablise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of cellular growth both in vitro and in vivo. Although the preferred method is to treat an individual in vivo it should nevertheless be understood that it may be desirable that the method of the invention may be applied in an in vitro environment, for example to facilitate the generation of cell lines. In another example, the application of the method of the present invention in an in vitro environment may extend to providing a readout mechanism for screening technologies such as those hereinbefore described. That is, molecules identified utilising these screening techniques can be assayed to observe the extent and/or nature of their functional effect on cellular growth.

Modulation of said activin β_{C} subunit functional levels is achieved via the administration of activin β_{C} subunit, a nucleic acid molecule encoding activin β_{C} subunit or an agent which effects modulation of activin β_{C} subunit activity or activin β_{C} subunit gene expression (herein collectively referred to as “modulatory agents”). Preferably, the subject method is utilised to down-regulate the cellular growth of a neoplastic cell in a mammal.

Accordingly, in one embodiment there is provided a method of down-regulating the growth of a neoplastic cell in a mammal, said method comprising administering to said mammal an effective amount of an agent for a time and under conditions sufficient to induce a functionally ineffective level of activin β_{C} subunit.

In another embodiment there is provided a method of down-regulating the growth of a neoplastic cell in a mammal, said method comprising administering to said mammal an effective amount of an agent for a time and under conditions sufficient to induce a functionally effective level of activin β_{C} subunit.

Reference to “induce” should be understood as a reference to achieving the desired activin β_{C} subunit level, whether that be a functionally effective level or a functionally ineffective level. Said induction is most likely to be achieved via the up-regulation or down-regulation of activin β_{C} subunit expression, as hereinbefore described, although any other suitable means of achieving induction are nevertheless herewith encompassed by the method of the present invention.

As detailed hereinbefore, a further aspect of the present invention relates to the use of the invention in relation to the treatment and/or prophylaxis of disease conditions or other unwanted conditions.

The present invention therefore contemplates a method of therapeutically and/or prophylactically treating a condition, or a predisposition to the development of a condition, characterised by an aberrant, unwanted or otherwise inappropriate level or bioactivity of activin β_{C} subunit in a mammal, said method comprising modulating the level of activin β_{C} subunit in said mammal.

Preferably, said activin β_{C} subunit levels or bioactivity are modulated intracellularly.

Reference to “aberrant, unwanted or otherwise inappropriate” activin β_{C} subunit levels should be understood as a reference to excessive levels, inadequate levels or to physiologically normal levels which are inappropriate in that they are unwanted or otherwise inappropriate.

Without limiting the present invention in any way, the disease or condition may include diseases or conditions of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum, oesophagus, myometrium and soft tissue.

Most particularly, said condition is a neoplastic condition.

Accordingly, the present invention preferably contemplates a method of therapeutically and/or prophylactically treating a neoplastic condition, or a predisposition to the development of a neoplastic condition, said method comprising modulating the level or bioactivity of activin β_{C} subunit wherein down-regulating said activin β_{C} subunit level to a functionally ineffective level inhibits abnormal cell growth.

Still more preferably, the present invention contemplates a method of therapeutically and/or prophylactically treating a neoplastic condition, or a predisposition to the development of a neoplastic condition, said method comprising administering an effective amount of an agent for a time and under conditions sufficient to induce a functionally ineffective level of activin β_{C} subunit.

Preferably, said agent is an antibody

Preferably, said neoplastic condition is a malignant neoplasia of the pancreas, brain and neural tissue, adrenal gland, thyroid gland, stomach, colon,, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum or oesophagus, myometrium and soft tissue or a non-malignant neoplasia of the fallopian tube, uterus, tonsil, spleen, appendix, seminal vesicle, myometrium and soft tissue.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome. For example, one may combine the method of the present invention with radiotherapy or chemotherapy.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of an agent capable of modulating the functionally effective level of activin β_{C} subunit in the manufacture of a medicament for the treatment of a condition characterised by an aberrant, unwanted or otherwise inappropriate level of activin β_{C} subunit.

In one aspect the present invention relates to the use of an agent as hereinbefore described in the manufacture of a medicament for the regulation of the abnormal growth of a cell wherein down-regulating activin β_{C} subunit to a functionally ineffective level inhibits abnormal growth.

In another aspect the present invention relates to the use of an agent as hereinbefore described in the manufacture of a medicament for the regulation of the abnormal growth of a cell wherein down-regulating activin β_{C} subunit to a functionally effective level inhibits abnormal growth.

Preferably, said neoplastic condition is a malignant neoplasia of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum or oesophagus, myometrium and soft tissue or a non-malignant neoplasia of the fallopian tube, uterus, tonsil, spleen, appendix, seminal vesicle, myometrium and soft tissue.

In yet another further aspect, the present invention contemplates a pharmaceutical composition comprising the modulatory agent as hereinbefore defined together with one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding activin β_{C} subunit or a modulatory agent as hereinbefore defined. The vector may, for example, be a viral vector.

Further features of the present invention are described in the following non-limiting Figures and/or Examples.

### EXAMPLE 1

### Detection of Activin AC Heterodimers in Biological Samples

Activin AC Enzyme Linked Immunosorbent Assay (ELISA)

Plates were coated and blocked as previously described (Evans et al, 1998,. J Endocrinol 156:275-82) with human activin β_{C} subunit Clone I monoclonal antibody on 96-well ELISA plates (MaxiSorp; Nunc, Roskilde, Denmark). bFF was used as an interim standard. The top dose in the assay, equivalent to a 1/10 dilution, was assigned the arbitrary unitage of 10 U/ml. Standards and samples were diluted in DMEM/5% FCS, as used in the culture experiments. 125 μl of a 6% sodium dodecyl sulphate (SDS) solution in PBS was added (3% final concentration, w/v) to 125 μl of sample or standard, mixed, boiled for 3 minutes and allowed to cool. The addition of PBS to the SDS solution was found to improve the performance of the assay and the linearity of the dose-response curve of the standard and samples. Thereafter, 20 μl of 30% H₂O₂ (2% final concentration, v/v) was added and the tubes incubated at room temperature for 30 mins. To each well, was added 25 μl of 20% BSA/0.1 M Tris/0.9% NaCl/5% Triton X-100/0.1% sodium azide prior to the addition of 100μl duplicates of the treated samples. Plates were incubated overnight in a sealed humidified box. The next day, the plates were washed with 0.05M Tris/0.9% NaCl/0.05% Tween-20/0.1% NaN₃ before 50 μl biotinylated E4 monoclonal antibody directed to the activin β_{A} subunit in 5%BSA/0.1M Tris/0.9% NaCl/5% Triton X-100/0.1% sodium azide was added to each well and incubated for 2 hours at room temperature. After washing, alkaline phosphatase linked to streptavidin (Invitrogen Corporation, Carlsbad, Calif.) was added to the wells and incubated at room temperature for one hour. After further washes, the alkaline phosphatase activity was detected using an amplification kit (ELISA Amplification System; Invitrogen) whereby the substrate was incubated for one hour at room temperature, followed by the addition of an amplifying reagent. The reaction was stopped with the addition of 50 μl of 0.4M H₂SO₄. The plates were read at 492 nm with a 630 nm reference filter on a Multiskan RC plate reader (Labsystems, Helsinki, Finland) and data were processed using Genesis Lite EIA software (Labsystems).

Activin AC Heterodimer Formation, In Vivo

Activin AC protein levels (U/ml) were measured in samples of human serum. (see Table 2).

### EXAMPLE 2

### Activin β_{C} Subunit Protein Immunohistochemistry in Normal/Diseased Human Tissues and Animal Tissues

Human Tissues

Human normal tissue array (AA) and human tumor tissue array (BB) were obtained from SuperBioChips Laboratories (Seoul, Korea). Tissue from patients with breast cancer, colon cancer, gastric cancer, skin cancer, menigioma, schwanoma, bladder cancer and thyroid cancer were obtained.

Animal Tissues

The left sagittal brain was removed from a transgenic mouse with a neurodegenerative disorder (familial amyotrophic lateral sclerosis) and corresponding wild type animals (38). The tissue was fixed in 4% paraformaldehyde, processed to paraffin and 3 μm tissue sections were cut.

Ewes were killed by i.v. injection of 20 ml of Lethobarb (Virbac, Peakhurst, NSW, Australia). The heads were then perfused with 21 ml of heparinized saline followed by 11 ml of 10% formalin fixative solution and 0.51 ml of the same fixative solution containing 20% sucrose. The brain blocks were left overnight in the same fixative containing 30% of sucrose and then in 30% sucrose in PBS until they sank. The brain blocks were then frozen in dry ice, wrapped parafilm and stored at −20° C. until sectioning. Coronal sections (7 μm) of sheep pituitary were cut on a cryostat, thaw mounted onto superfrost slides and stored at −20° until used. Coronal sections of sheep brain (40 μm) were cut on a cryostat, collected into individual tissue culture wells containing cryoprotectant and stored at −20° C. until used.

After being de-paraffinated the tissue underwent a pretreatment step of microwave heating in 0.1M glycine (pH 4.5). The sections were immunostained for activin β_{C} subunit protein using the DAKO Autostainer (DAKO, Carpinteria, USA). Briefly, endogenous peroxidase was blocked by incubation of sections with 0.03% H₂O₂ for 5 minutes (DAKO, Carpinteria, USA). After incubation with CAS Blocking solution (Zymed, Calif., USA) for 10 minutes, the sections were incubated with activin β_{C} antibody (working concentration 0.45 μg/ml) for 60 minutes. The antibody was detected by incubation with Envision polymer-anti-mouse-horse radish peroxidase (DAKO, Carpinteria, USA) for 15 minutes and visualised by reaction with diaminobenzidine (DAB) (DAKO, Carpinteria, USA) for 5 minutes. The specificity of immunostaining was examined by pre-incubation of primary antibody with 100-fold (w/w) excess of corresponding activin β_{C} subunit peptide.

Immunolocalisation of Activin β_{C} Subunit Protein in Normal and Diseased Human Tissues

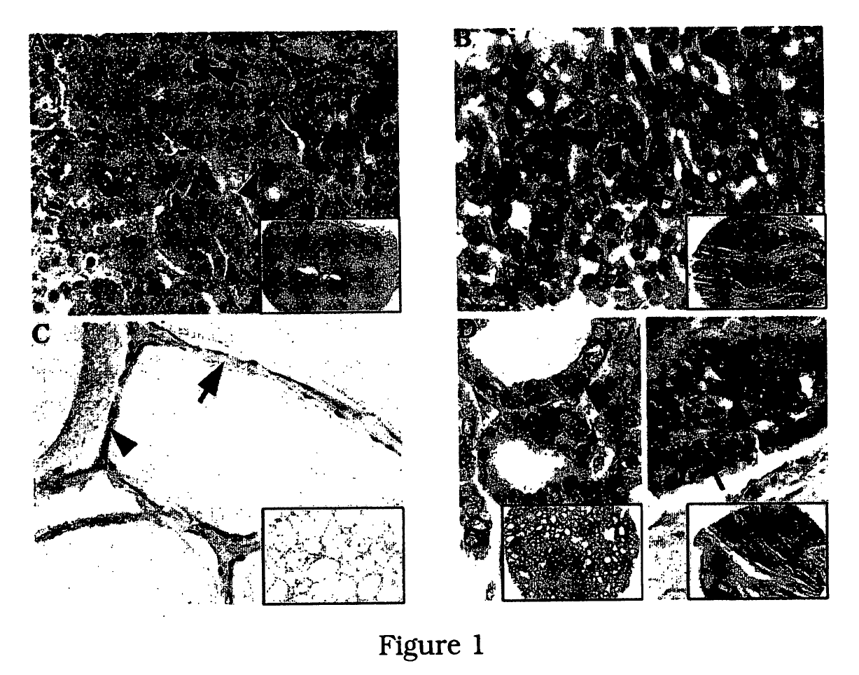

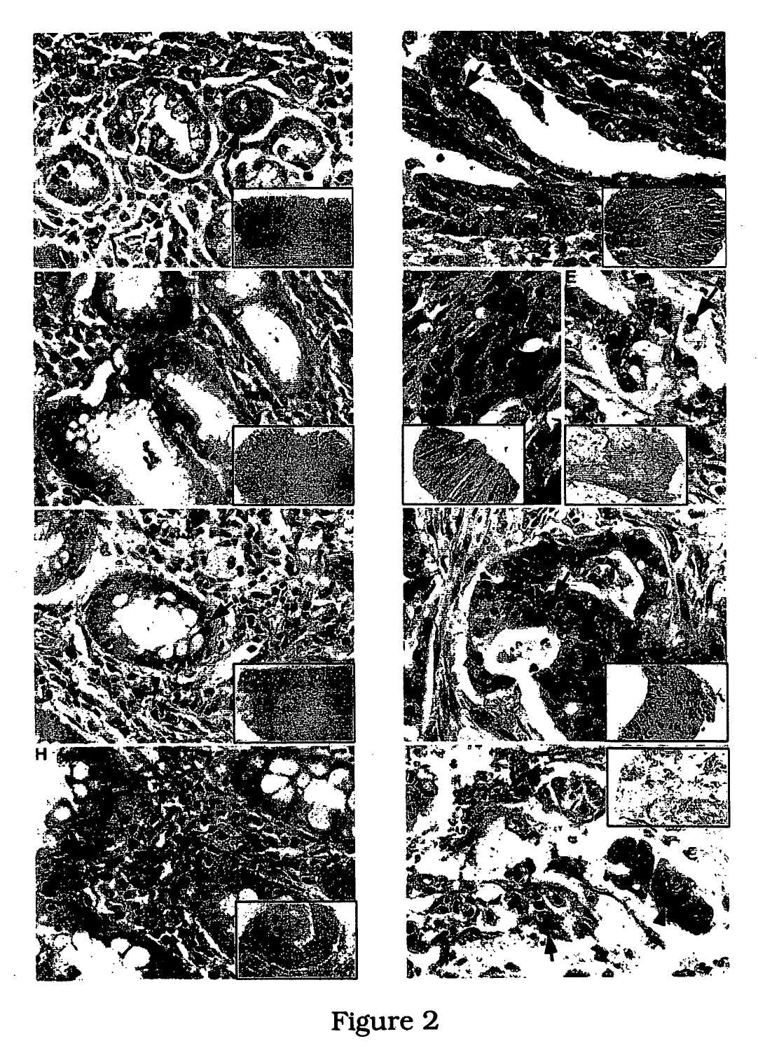

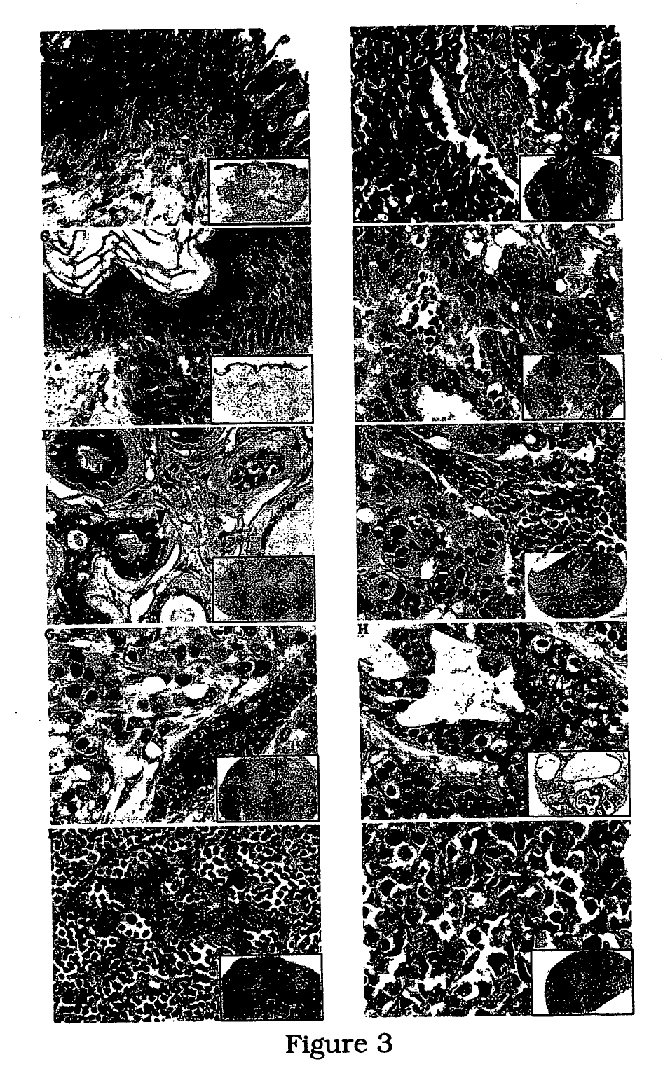

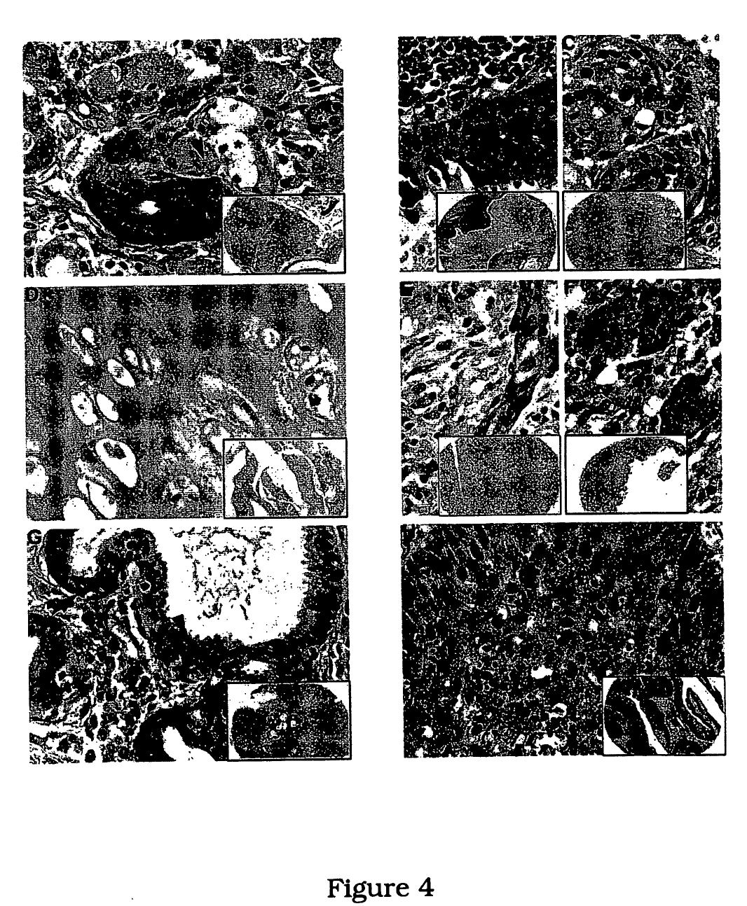

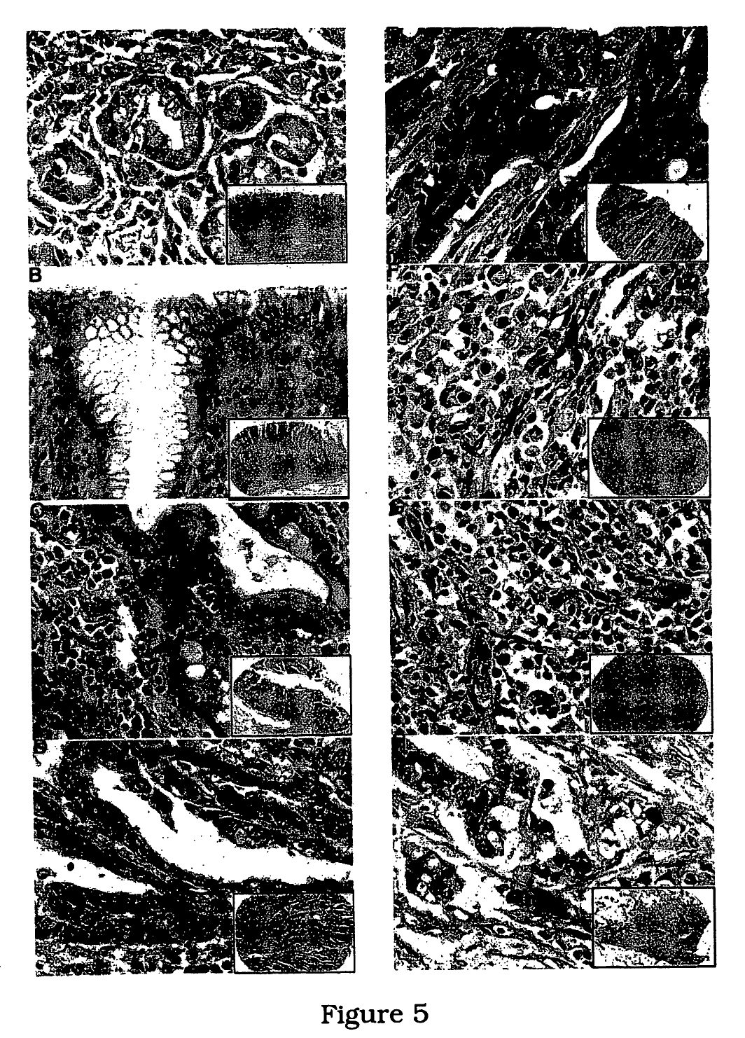

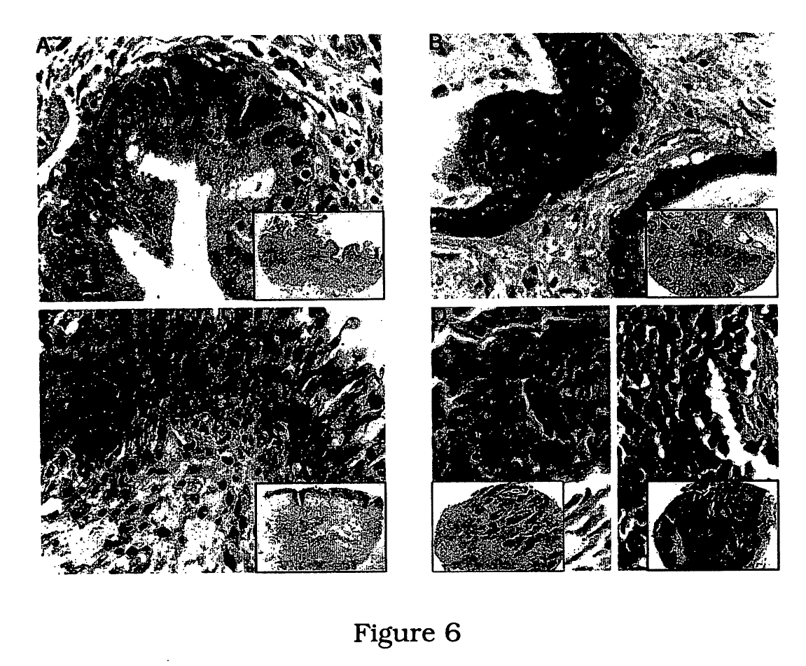

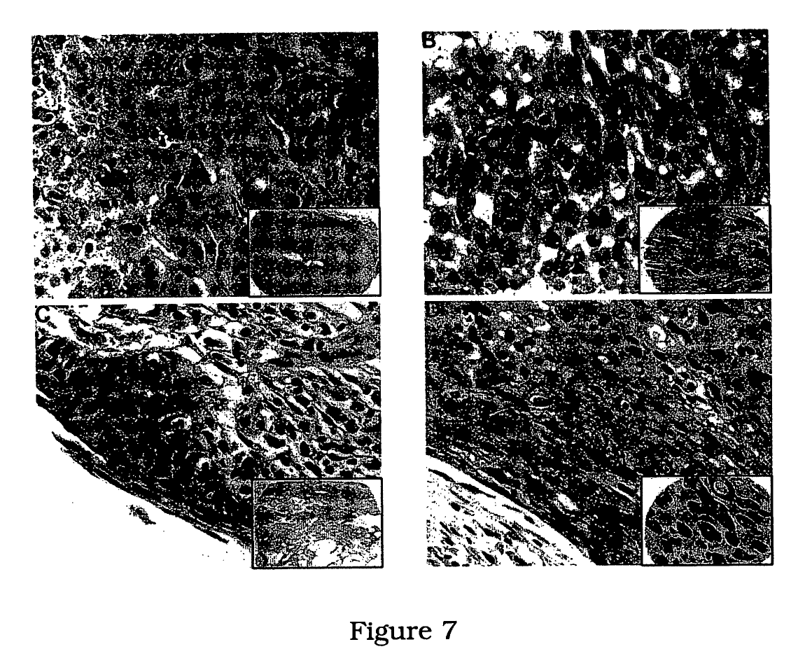

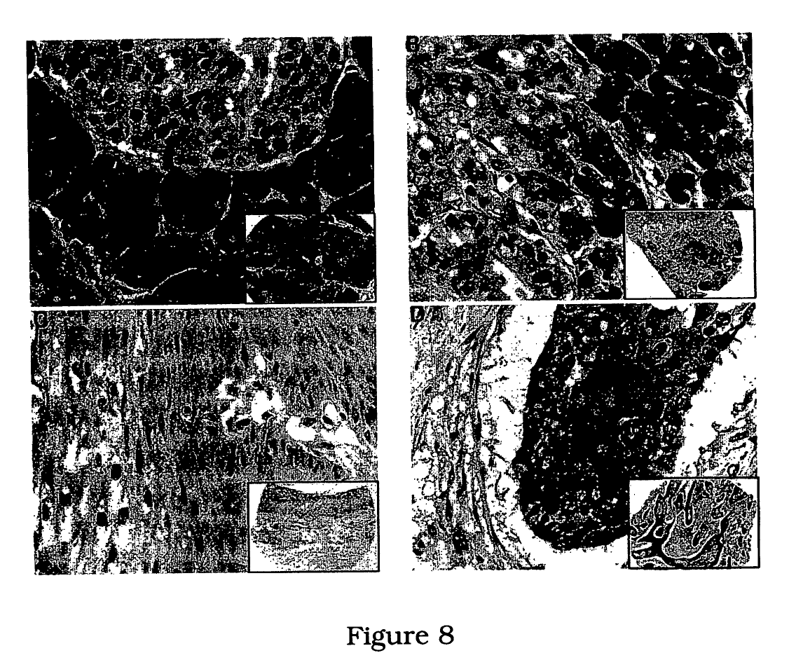

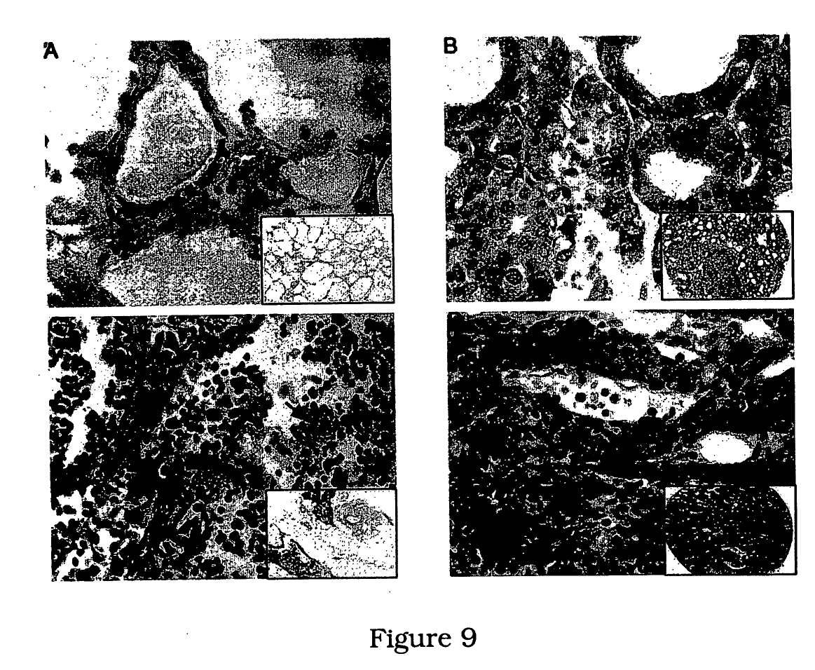

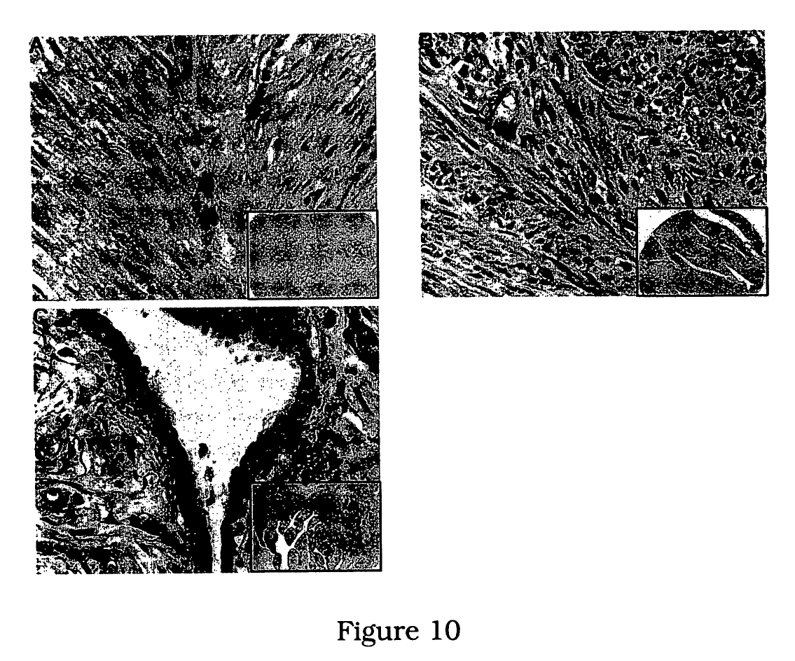

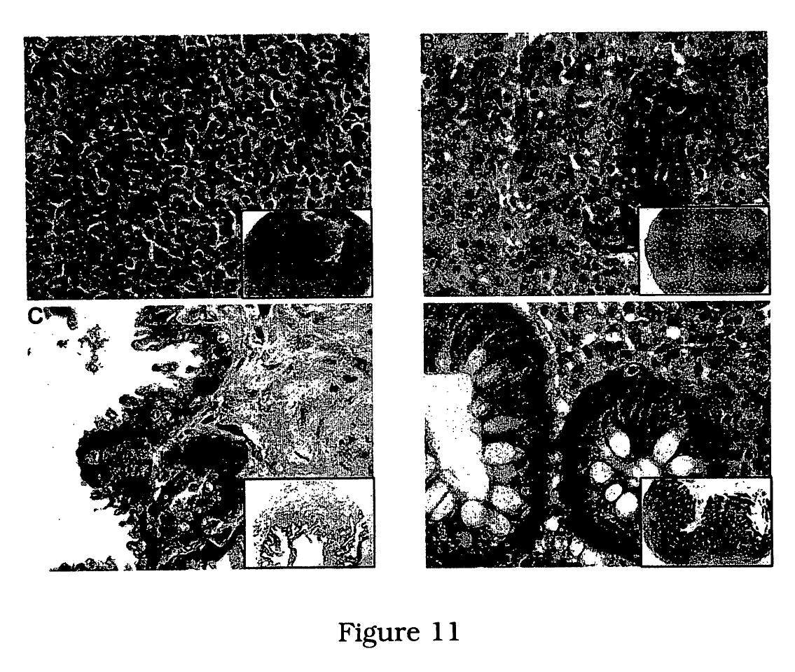

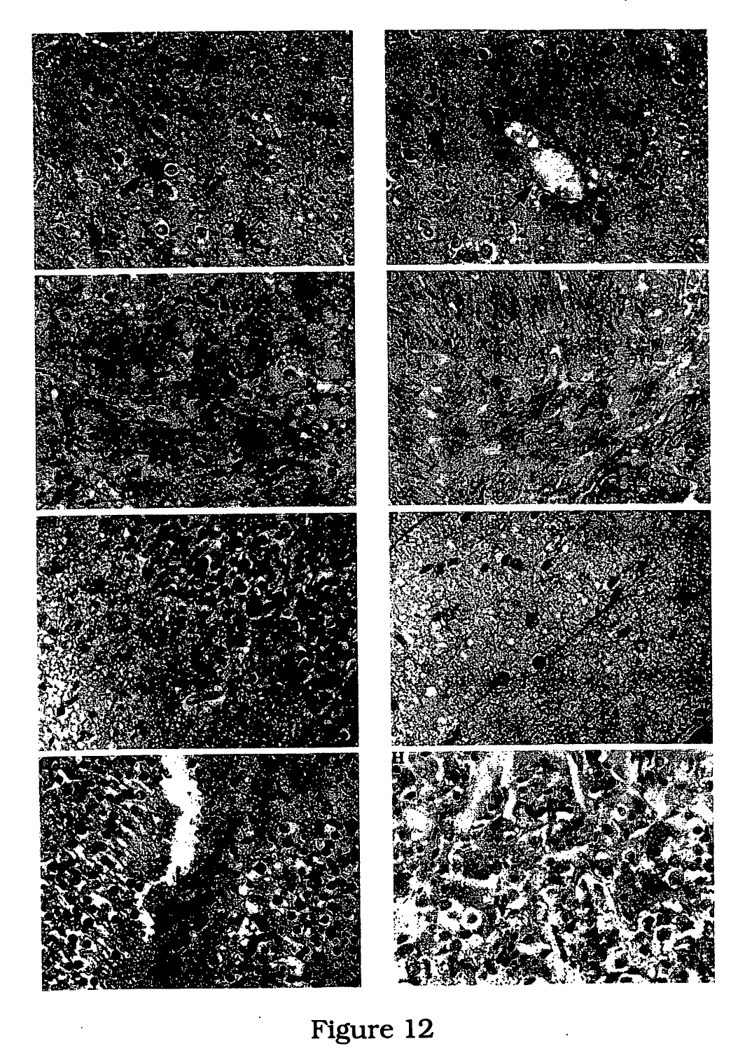

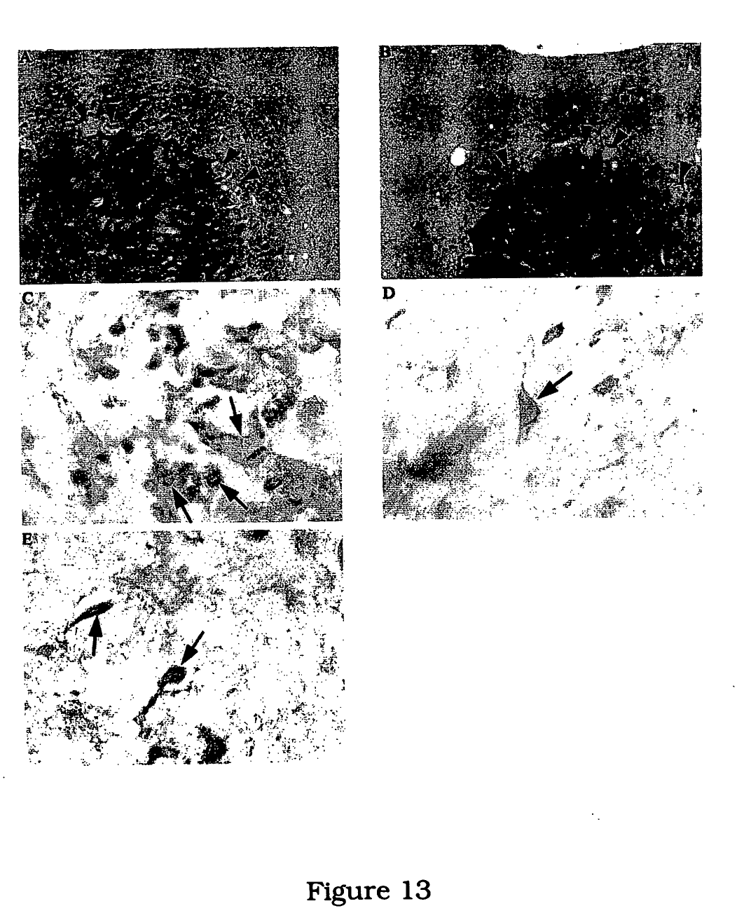

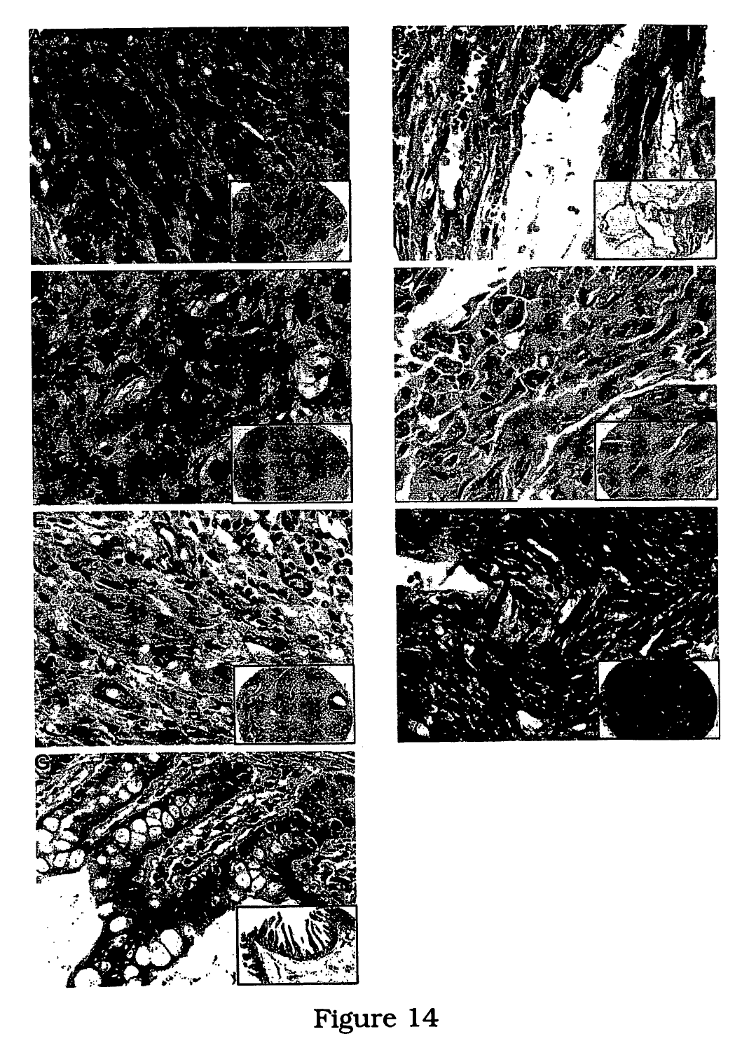

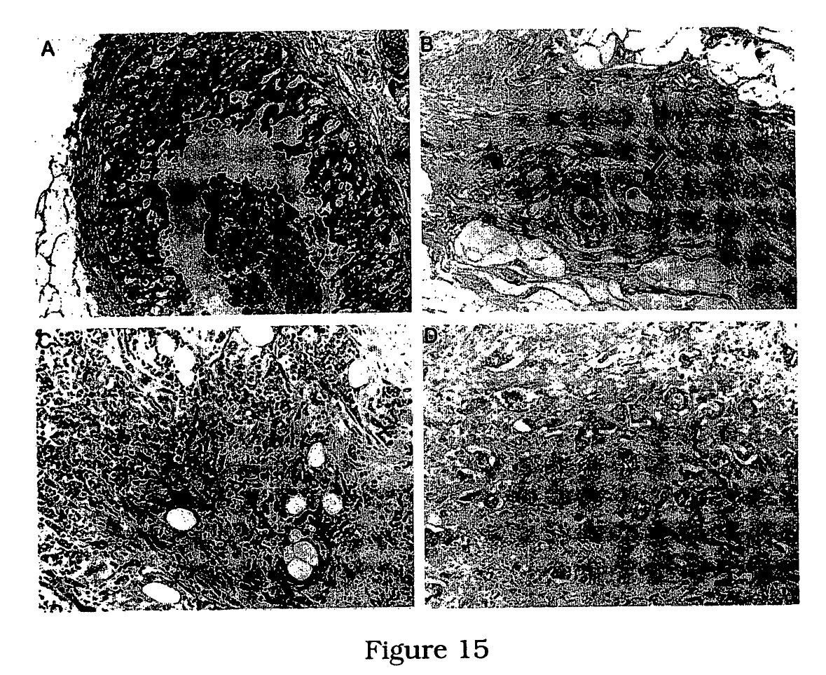

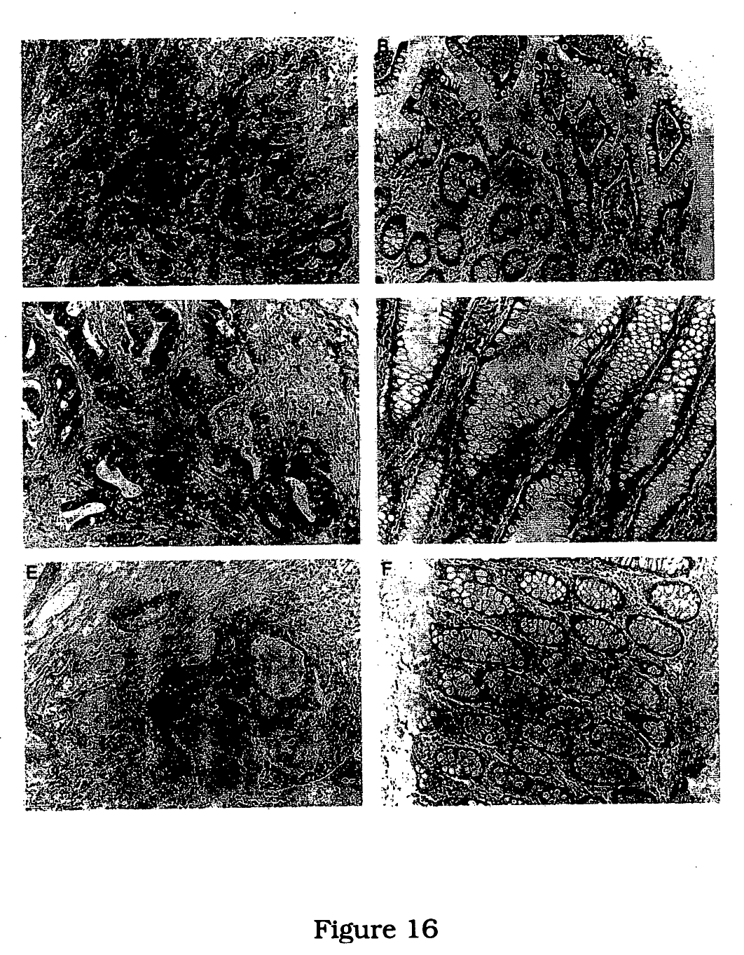

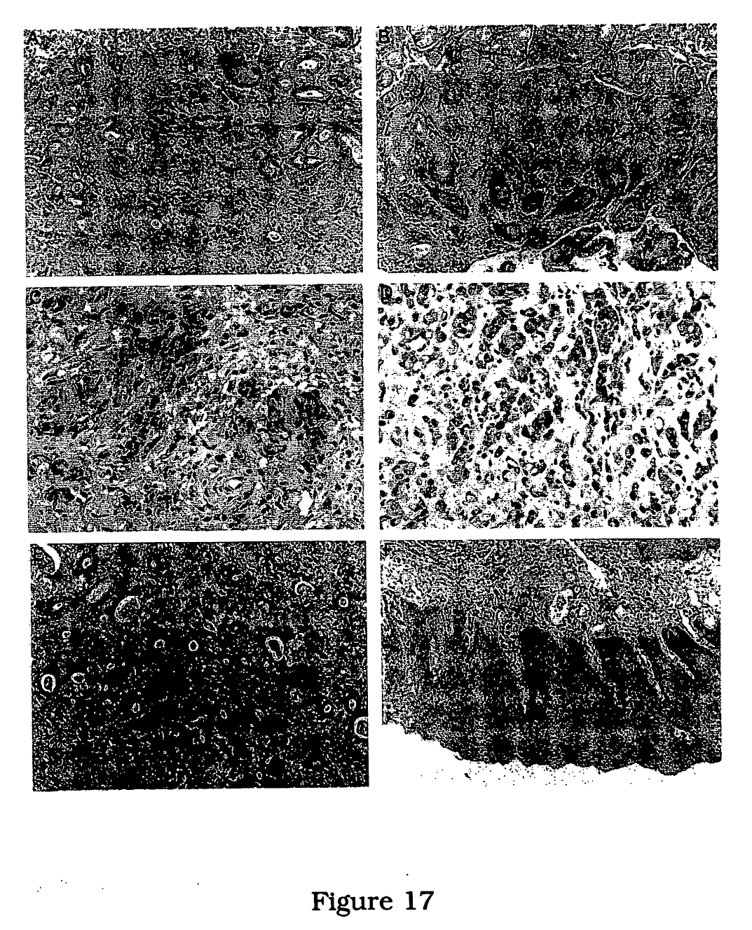

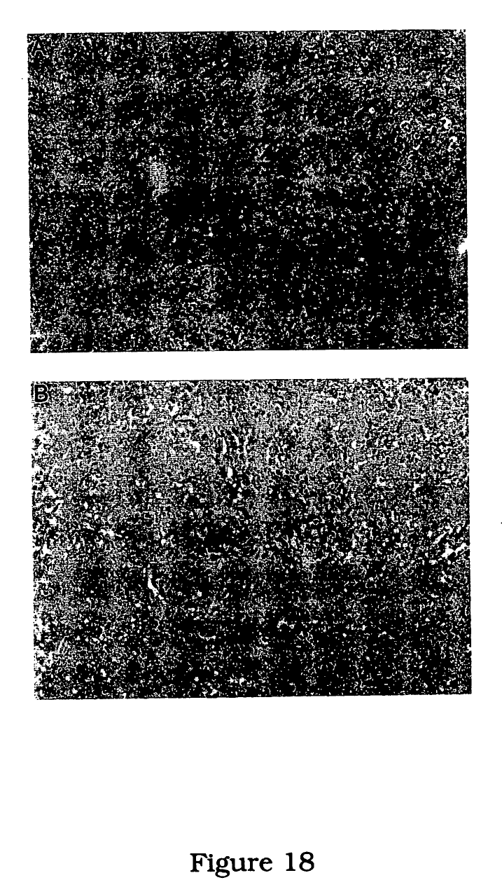

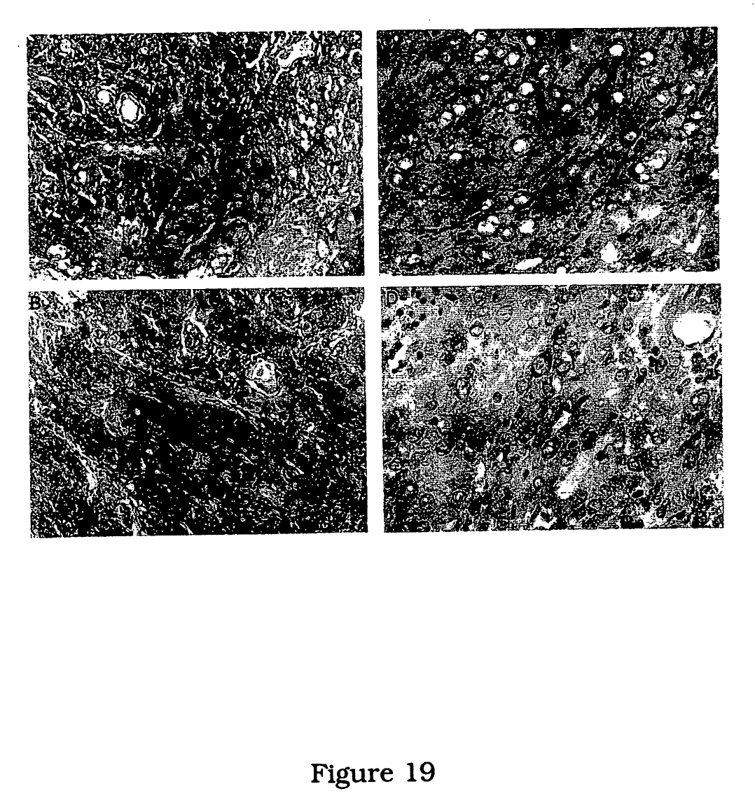

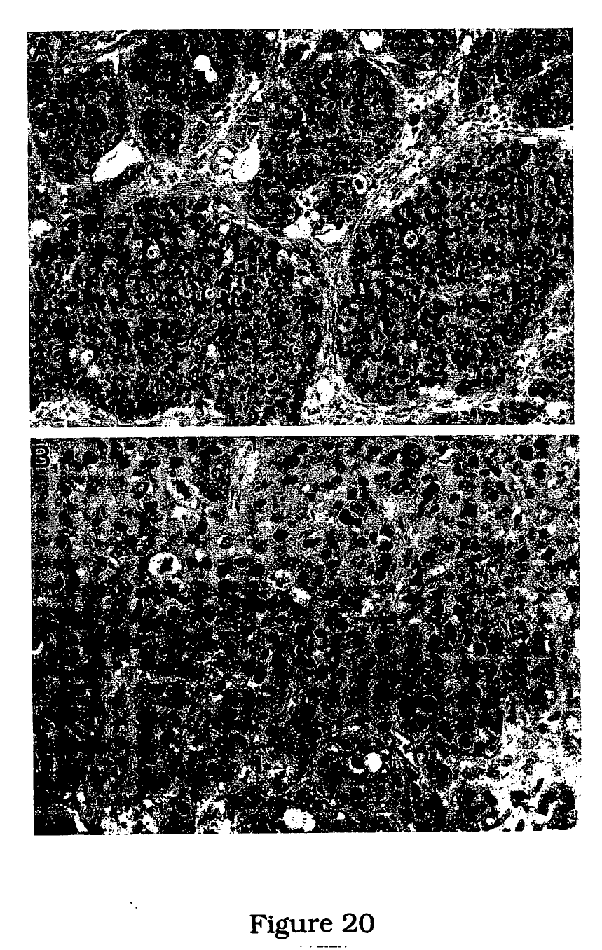

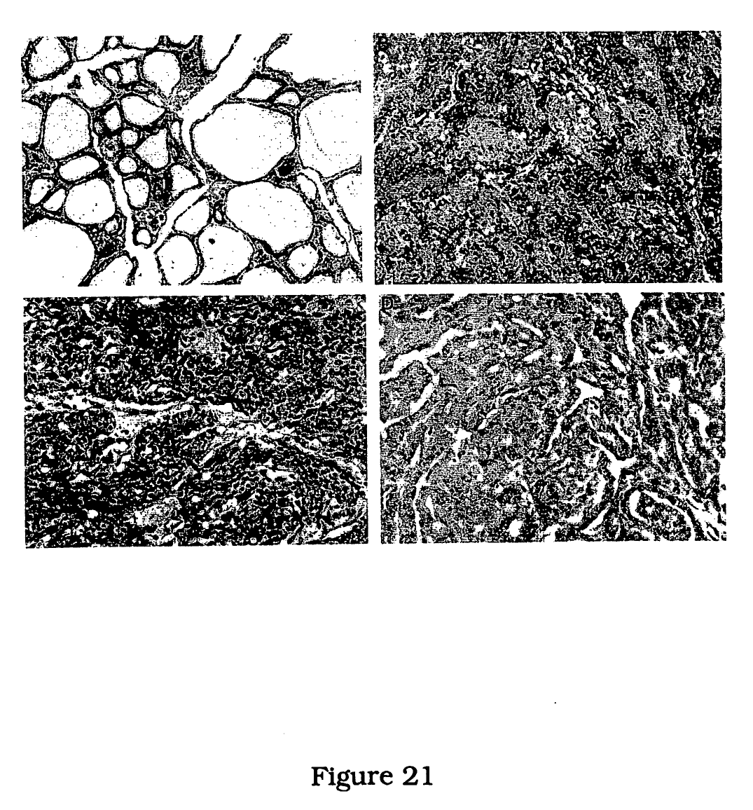

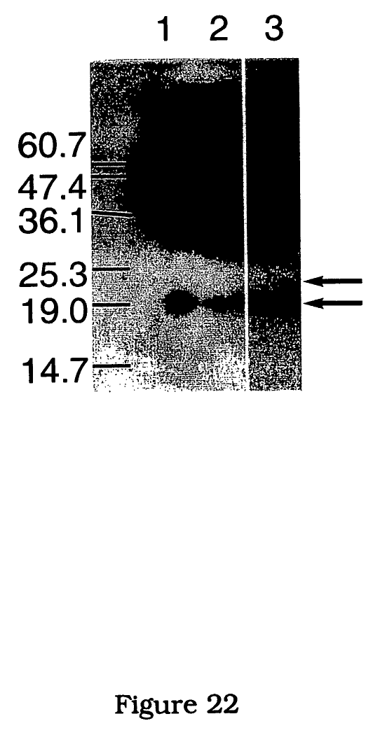

The activin β_{C} subunit protein was demonstrated to immunolocalise to most benign and malignant human organs studied. Both cytoplasmic and/or nuclear staining was commonly observed and changes in these patterns occurred between the benign and malignant state. FIGS. 1-14 fully describe the staining pattern and the descriptions below indicate some of the significant findings.

The adrenal gland and thyroid gland, as shown in FIG. 1, displayed strong activin β_{C} subunit protein localisation in malignancy. Staining in the adrenal and thyroid glands shows increased intensity in a malignant state.

Most of the adenocarcinomas of the stomach, colon and rectum (FIG. 2) showed a pattern of both cytoplasmic and nuclear activin β_{C} subunit localisation. This staining pattern differed to the variable and predominantly cytoplasmic staining observed in the normal stomach, colon, and rectum.

Strong activin β_{C} subunit protein nuclear staining became apparent in the development of malignancy in the skin, breast and lymph node (FIG. 3). Some nuclear staining was observed in some cells of the benign skin and breast, however stronger staining was displayed in malignant tissue. Similarly, cytoplasmic localisation of activin β_{C} subunit protein was observed in the normal salivary gland and nasal cavity however this staining showed strong nuclear localisation, as well as cytoplasmic, in malignancy (FIG. 4). In addition, little staining was observed in chondrosarcoma (a benign condition of the bone), however strong nuclear and cytoplasmic activin β_{C} subunit protein localisation was observed in malignancy. The normal stomach (FIGS. 2 and 5) displayed variable cytoplasmic localisation. In addition to the stomach adenocarcinomas described above, other stomach malignancies displayed nuclear localisation (and stromal localisation) including stomach signet ring cell carcinoma, stomach lymphoma and metastatic stomach carcinoma. The normal bladder has little nuclear staining however following the development of cancer, strong nuclear staining was observed (FIG. 6).

Some organs, such as the gallbladder, adrenal, uterine cervix, and pancreas had varying degrees of nuclear and cytoplasmic staining in the benign and malignant state (FIGS. 6, 7, 8).

The oesophagus, thyroid and thymus showed little or no staining in the normal tissue, however following the development of cancer increased activin β_{C} subunit protein localisation was observed (FIGS. 8, 9).

Other tissues that immunolocalised activin β_{C} subunit protein included the myometrium, fallopian tube, tonsil, spleen, appendix and seminal vesicle as well as benign disorders of the uterus (FIGS. 10 and 11).

Normal, damaged and malignant skin immunolocalised different patterns of activin β_{C} subunit protein staining. Both nuclear and cytoplasmic staining was observed in the normal skin and tumours including squamous cell and melanoma (FIGS. 3 and 14).

The normal breast immunolocalised the activin β_{C} subunit and different breast tumours (residual infiltrating duct carcinoma, breast infiltrating lobular carcinoma, breast papillary carcinoma) also displayed cytoplasmic or nuclear localisation (FIG. 3).

The brain displays strong activin β_{C} subunit protein localisation in both the benign and malignant disorders. In particular, astrocytes, blood brain barrier and neurons strongly localise activin β_{C} subunit (FIG. 12). The endocrine cells of the sheep and human pituitary and the neuronal cells of the cerebellum, pre-optic area and hypothalamus display activin β_{C} subunit localization (FIG. 13). Strong localisation is also observed in tumour cells of the brain, in particular tumour cells in (I) glioblastoma of two patients and (II) meningioma of four patients (FIG. 12).

### EXAMPLE 3

### Breast Tissue Analysis

Activin β_{C} subunit protein localisation was investigated in 10 patients with breast cancer (FIG. 15) as classified by the accepted classification system BRE [BRE grade I (low grade) to BRE grade 3 (high grade)]. Analysis of these patient tissues showed low levels of activin β_{C} subunit protein in benign breast tissue, variable staining in different grades (low, intermediate, high) of in situ (intraduct carcinoma), variable staining in BRE grades 1-3 infiltrating lobular or ductal breast cancer and no localisation in mucinous breast cancer. As shown in Table 3, two patients with BRE Grade 2 infiltrating lobular carcinoma displayed either positive or negatively stained tumor cells. One patient with BRE Grade 3 infiltrating ductal carcinoma had tumour cells that immunolocalised activin β_{C} subunit protein in the nucleus, other patients with BRE Grade 2 or 3 had intermittent or no staining in infiltrating ductal carcinoma cells. Areas of intraductal carcinoma within these patient tissues had either no staining (low and high grade in situ) or areas of positive cell staining (intermediate in situ). One patient classified with high grade in situ (with no invasive component) showed strong immunolocalisation of activin β_{C} subunit protein in intraductal cells.

### EXAMPLE 4

### Colon Tissue Analysis

Activin β_{C} subunit protein localisation was investigated in nine patients with colon cancer (FIG. 16). As shown in Table 4, patients with poorly differentiated colonic adenocarcinoma have either uniform strong cytoplasmic staining for activin β_{C} subunit protein in tumour cells (as seen in one patient) or tumour cells with both strong positive and negative staining as observed in three other patients. Two patients with moderately differentiated colonic adenocarcinoma showed tumour cells with both positive and negative staining, while tumour cells in two other patients displayed no staining for activin β_{C} subunit protein. One patient with a mucinous subtype of colon adenocarcinoma did not immunolocliase the activin β_{C} subunit. In the patients with positive tumour cell staining, an increase in activin β_{C} subunit protein intensity was observed in colon adenocarcinomas when compared to weaker staining in the adjacent non-malignant areas.

### EXAMPLE 5

### Gastric Tissue Analysis

Activin β_{C} subunit protein localisation was investigated in six patients with gastric cancer (FIG. 17). As shown in Table 5, normal gastric mucosa showed little activin β_{C} subunit protein staining. In patients with intestinal type of gastric adenocarcinoma, well/moderately well differentiated tumour cells did not immunolocalise the activin β_{C} subunit protein while two patients with poorly differentiated tumour cells had either strong cytoplasmic or variable nuclear staining. In two patients with signet ring cell type of gastric adenocarcinoma, tumour cells displayed either variable nuclear staining or no localisation of activin β_{C} subunit protein.

### EXAMPLE 6

### Skin Analysis

Activin β_{C} subunit protein localisation was investigated in five patients with metastatic melanoma within brain (FIG. 18). As shown in table 6 two patients with metastatic melanoma immunolocalised activin β_{C} subunit protein in the cytoplasm of tumour cells, one patient had variable tumour cell staining and two patients had no localisation in the melanoma tumour cells.

### EXAMPLE 7

### Brain Tissue Analysis

As shown in table 7, strong activin β_{C} subunit protein localisation was seen in brain tissue of 17 patients with meningioma and three patients with Schwanoma (FIG. 19). Activin β_{C} subunit protein was predominantly localised as strong uniform cytoplasmic staining in meningioma of patients with WHO grade 1 classification, however one patient had very strong cell membrane localisation. Two meningioma patients classified as WHO grade 2 had focal weak activin β_{C} subunit protein immunoreactivity in some regions of tumour cells when compared to other tumor cells within the tissue which strongly localized activin β_{C} subunit protein. All patients with Schwanoma displayed strong uniform cytoplasmic staining of activin β_{C} subunit protein in the tumour cells.

### EXAMPLE 8

### Bladder Tissue Analysis

Activin β_{C} subunit protein localisation was investigated in three patients with bladder cancer (FIG. 20). As shown in table 8, patients with high grade (grade 3) transitional cell carcinoma immunolocalise activin β_{C} subunit protein in the cytoplasm and nuclei of the tumour cells. One patient with transitional cell carcinoma did not localise activin β_{C} subunit protein.

### EXAMPLE 9

### Thyroid Tissue Analysis

Activin β_{C} subunit protein localisation was investigated in four patients with thyroid cancer (FIG. 21). In regions of the normal thyroid, the follicular epithelium displayed intermittent cytoplasmic staining. As shown in table 9, two patients with papillary cancer immunolocalised activin β_{C} subunit protein in the cytoplasm of tumour cells, however staining was variable and not observed in all tumour cells. Patients with medullary and follicular cancer had consistent staining of activin β_{C} subunit protein in the cytoplasm of tumour cells.

### EXAMPLE 10

### Normal Human Serum Analysis

Western Blot methodology determining activin β_{C} subunit protein dimers in normal human serum

SDS-page was performed under non reducing conditions using 15% polyacrylamide gel. Human normal serum samples were diluted 1:3 with 4% SDS, boiled at 100° C. in heat block for 5 minutes and supernatants were collected. The serum samples, treated with 4% SDS, were diluted in 1:2 in sample buffer (Tris, 1% SDS, glycerol, and 0.01% bromophenol blue, pH 7.2). Samples were incubated at 100° C. in a heat block for 5 min, centrifuged briefly, 20 ul of samples was loaded on gel and gel was run at 150 Volts for 90 min with running buffer (Tris, glycin, 10% SDS). Immobilon P (PVDF) membrane, which had been pre-incubated in methanol for 15 sec, and milliQ water for 2 min, was equilibrated along with the gel in transfer buffer (0.7 molecule/L glycin, 0.3 molecule/L Tris and 15.6% ethanol) for 5 min. The proteins in the gel were transferred to the membrane at 100 Volts for 60 minutes. Following transfer, the membrane was soaked in 1×TBS with 0.05% Tween 20 for 5 min. The membrane was blocked (5% Non-fat milk powder, 0.05% Tween in 1×TBS) for 90 minutes. Activin beteac clone 1 antibody was added at 1:6000 in 5% milk in 1×TBS overnight at 4° C. Following washing, the membrane was incubated with goat anti-mouse HRP 1:10,000 in 5% milk in TBS for 45 minutes at RT. After subsequent washes, ECL plus substrate (Amersham Bioscience UK Limited, UK) was added according to manufacturer's instructions. The membrane was placed in an x-ray cassette and exposed to X-Omat film (Kodak).

Results

Using a specific monoclonal antibody to the activin β_{C} subunit, Western blot analysis showed samples of normal human serum from two people contained dimeric activin β_{C} proteins at sizes of approximately 20 kDa, predicted to be activins C or BC. One person had a strong 20 kDa band indicating the presence of either activin C or BC, as well as a weaker 23 kDa band indicating that another activin β_{C} dimer, activin AC, was present in the serum (as shown in FIG. 22).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2

| Sample | |
|---|---|
| Normal serum | Activin AC (U/ml) |
| Male serum control | 0.033 |
| Female serum control | 0.038 |

TABLE 3

| | Total | | | |
|---|---|---|---|---|
| Cancer | patients | +ve cancer | +/−cancer | −ve cancer |
| Breast carcinoma | 10 | 3 | 1 | 6 |
| Infiltating lobular | | | | |
| BRE grade 2 | | 1 | | 1 |
| Infiltrating ductal | | | | |
| BRE grade 2 | | | 1 | 2 |
| BRE grade 3 | | 1 (+ nuclear) | | 2 |
| In-situ carcinoma | | | | |
| High grade | | 1 (+++) | | |
| Mucinous | | | | 1 |

TABLE 4

| | Total | +ve | | |
|---|---|---|---|---|
| Cancer | patients | cancer | +/−cancer | −ve cancer |
| Colonic adenocarcinoma | 9 | 1 | 5 | 3 |
| Moderately well | | | 2 | 2 |
| differentiated | | | | |
| Poorly differentiated | | 1 (++) | 3 | |
| Mucinous subtype | | | | 1 |

TABLE 5

| | Total | +ve | | |
|---|---|---|---|---|
| Cancer | patients | cancer | +/−cancer | −ve cancer |
| Gastric adenocarcinoma | 6 | 1 | 2 | 3 |
| Intestinal type | | | | |
| well differentiated | | | | 1 |
| moderately well | | | | 1 |
| differentiated | | | | |
| poorly differentiated | | 1 (++) | 1 (+/− | |
| | | | nuclear) | |
| Signet ring cell type | | | 1 (+/− | 1 |
| | | | nuclear) | |

TABLE 6

| | Total | | | |
|---|---|---|---|---|
| Cancer | patients | +ve cancer | +/−cancer | −ve cancer |
| Melanoma | 5 | 2 | 1 | 2 |
| metastastic | | 1(+++), | 1 | 2 |
| within brain | | 1 (+) | | |

TABLE 7

| | Total | | | |
|---|---|---|---|---|
| Brain | patients | +ve cancer | +/−cancer | −ve cancer |
| Meningioma | 17 | | | |
| WHO grade 1 | | 15 | | |
| WHO grade 2 | | | 2 | |
| Schwanoma | 3 | 3 | | |

TABLE 8

| | Total | | | |
|---|---|---|---|---|
| Cancer | patients | +ve cancer | +/−cancer | −ve cancer |
| Bladder transitional | 3 | 1 | 1 | 1 |
| cell carcinoma | | | | |
| Grade 3 (high grade) | | 1 (+ | 1 | 1 |
| | | cyto/nuclear) | | |

TABLE 9

| | Total | | | |
|---|---|---|---|---|
| Cancer | patients | +ve cancer | +/−cancer | −ve cancer |
| Thyroid carcinoma | 4 | 2 | 2 | |
| Papillary | | | 2 | |
| Follicular | | 1 | | |
| Medullary | | 1 | | |

### BIBLIOGRAPHY

Bunin B A, et al., 1994, Proc. Natl. Acad Sci. USA, 91:4708-4712
Burke and Warren, 1984, Cell 36:847-56
Chang et al, 2001, Mol Cell Endocrinol. Jun 30;180(1-2):39-46
Chang et al, 2001, Mol Cell Endocrinol. Jun 30;180(1-2):39-46
Chen et al, 1996, Nature 383:691-6
Chen et al, 1997, Nature 389:85-9
Cole 1985 Monoclonal Antibodies and Cancer Therapy. In. Alan R. Liss, Inc: 77-96
de Kretser et al, 1999, J Endocrinol 161:195-8
DeWitt S H, et al., 1993, Proc. Natl. Acad. Sci. USA, 90:6909-6913
Esquela et al, 1997, Biochem Biophys Res Commun 235:553-6
Evans et al, 1998, J Endocrinol 156:275-82
Fang J et al, 1997, Biochem Biophys Res Commun. 231(3):655-61
Groome et al, 2001, J Mol. Cell. Endo. 180:73-77
Hotten G et al, 1995, Biochem Biophys Res Commun 206:608-13
Hubner et al, 1999, Histol Histopathol 14:295-304
Kozbar 1983 Immunology Today 4:72
Kron et al, 1998, J Virol Methods 72:9-14
Lau et al, 2000, Mol Cel Biol, 20 (16):6127-37
Lau et al, 1996, Biochim Biophys Acta 1307:145-8
Lebrun et al, 1999, Mol Endocrinol 13:15-23
Luisi et al, 2001, Eur J Endocrinol 145:225-36
McDowell et al, 1999, Semin Cell Dev Biol 10:311-7
McFarlane et al, 1996 Eur J Endocrinol 134:481-9
Morgan and Roth, 1988, Immunol Today 9:84-8
Moore, A., Basilion, J., Chiocca, E., and Weissleder, R., 1988, BBA, 1402:239-249
O'Bryan et al, 2000, J Mol Endocrinol 24:409-18
Oda et al, 1995, Biochem Biophys Res Commun 210:581-8
Pangas et al, 2000, Trends Endocrinol Metab 11:309-314
Schmitt et al, 1996, Genomics 32:358-66
Stein and Cohen, 1988 Cancer Res 48:2659-68
van der Krol et al., 1988 Biotechniques 6:958-976
Wedemeyer, N., Potter, T., Wetzlich, S. and Gohde, W. 2002, Clinical Chemistry 48:9 1398-1405
Weissleder, R., Moore, A., Ph.D., Mahmood-Bhorade, U., Benveniste, H., Chiocca, E. A., Basilion, J. P. 2000, Nature Medicine, 6:351-355
Wrana and Attisano, 2000, Cytokine Growth Factor Rev 11:5-13
Zhang et al, 1997, Endocr J 44:759-64

1. A method of detecting in a mammal the onset, or predisposition to the onset, of a condition characterised by modulation of the level or bioactivity of activin β_{c}, which level is modulated relative to normal levels, said method comprising screening for the level of activin β_{C} protein and/or gene expression in a biological sample derived from said mammal.

2. A method of monitoring for the onset or progression of a condition characterised by modulation of the level or bioactivity of activin β_{C} in a mammal, which level is modulated relative to normal levels, said method comprising screening for the level of activin β_{C} protein and/or gene expression in a biological sample derived from said mammal.

3. The method according to claim 1 or 2 wherein said activin β_{C} subunit is in monomeric form.

4. The method according to claim 1 or 2 wherein said activin β_{C} subunit is in dimeric form.

5. The method according to claim 4 wherein said activin β_{C} dimer is one or more of activin AC (β_{A}-β_{C}), activin BC (β_{B}-β_{C}), activin C (β_{C}-β_{C}), activin CD (β_{C}-β_{D}) or activin CE (β_{C}-β_{E}).

6. The method according to any one of claims 1-5 wherein said condition is a condition of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum, oesophagus, myometrium and soft tissue.

7. The method according to claim 6 wherein said condition is a malignant neoplasia.

8. The method according to claim 7 wherein said malignant neoplasia is a malignant neoplasia of the pancreas, brain and neural tissue, adrenal gland, thyroid gland, stomach, colon, bladder, endometrium, breast, lymph node, skin, salivary Gland, bone, nasal cavity, duodenum, gallbaldder, cervix, thymus, larynx, tongue, small intestine, rectum or oesophagus, seminal vesicle cancer, brain cancer, splenic cancer or soft tissue cancer.

9. The method according to claim 7 wherein said condition is a non-malignant neoplasia.

10. The method according to claim 9 wherein said non-malignant neoplasia is a neoplasia of the fallopian tube, uterus, tonsil, spleen, appendix, seminal vesicle, myometrium or soft tissue.

11. The method according to any one of claims 6-10 wherein said modulation is an increase in the level or bioactivity of activin β_{C} subunit.

12. The method according to any one of claims 6-10 wherein said modulation is a decrease in the level or bioactivity of activin β_{C} subunit.

13. The method according to claim 11 or 12 wherein the biological sample is selected from the group including serum, tissue extracts, body fluids, cell culture medium, extracellular medium, supernatants, biopsy specimens or resected tissue.

14. The method according to any one of claims 1-13 wherein the screening assay is directed to detecting the activin β_{C} subunit and is performed utilising an antibody directed to activin β_{C} subunit.

15. The method according to any one of claims 1-13 wherein said screening assay is directed to detecting activin β_{C} dimers and is performed utilising an antibody directed to the activin β_{C} subunit and one or more antibodies directed to one or more of the activin β_{A}, β_{B}, β_{C}, β_{D} or β_{E} subunits.

16. The method according to claim 15 wherein said activin β_{C} dimer is one or more of activing AC (β_{A}-β_{C}), activin BC (β_{B}-β_{C}), activin C (β_{C}-β_{C}), activin DC (β_{D}-β_{C}) or activin EC (β_{C}-β_{E}).

17. The method according to any one of claims 14-16 wherein said antibody recognises an epitope of activin β_{C} comprising the amino acid sequence: VPTARRPLSLLYYDRDSNIVKTDIPDMVVEAC (SEQ ID NO:1) or equivalent thereof.

18. A method of detecting the onset, predisposition to the onset, or monitoring for the onset or progression, of a condition characterised by modulation of the level of activin β_{C} in a mammal, which level is modulated relative to normal levels, said method comprising:
(a) contacting a first antibody that recognises an epitope of a first activin β subunit with a biological sample derived from said mammal;
(b) allowing the first antibody to bind to said first activin β subunit in said sample;
(c) washing said sample to substantially remove unbound material;
(d) contacting said sample with a second antibody that recognises an epitope of a second activin β subunit, wherein the second antibody is tagged with a labelling agent; and
(e) detecting the labelling agent to identify an activin β_{C} subunit dimer in said sample, wherein the first or second antibody recognises an epitope of an activin β_{C} subunit.

19. The method according to claim 18 wherein said activin β_{C} dimer is one or more of activin AC (β_{A}-β_{C}), activin BC (β_{B}-β_{C}), activin C (β_{C}-β_{C}), activin CD (β_{C}-β_{D}) or activin CE (β_{C}-β_{E}).

20. The method according to any one of claims 18-19 wherein said condition is a condition of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum, oesophagus, myometrium and soft tissue.

21. The method according to claim 20 wherein said condition is a malignant neoplasm.

22. The method according to claim 21 wherein said malignant neoplasm is a malignant neoplasm of the pancreas, brain and neural tissue, adrenal gland, thyroid gland, stomach, colon, bladder, endometrium, breast, lymph node, skin, salivary Gland, bone, nasal cavity, duodenum, gallbaldder, cervix, thymus, larynx, tongue, small intestine, rectum or oesophagus, seminal vesicle cancer, brain cancer, splenic cancer or soft tissue cancer.

23. The method according to claim 20 wherein said condition is a non-malignant neoplasm.

24. The method according to claim 23 wherein said non-malignant neoplasm is a neoplasm of the fallopian tube, uterus, tonsil, spleen, appendix, seminal vesicle, myometrium or soft tissue.

25. The method according to any one of claims 20-24 wherein said modulation is an increase in the level or bioactivity of activin β_{C} subunit.

26. The method according to any one of claims 20-24 wherein said modulation is a decrease in the level or bioactivity of activin β_{C} subunit.

27. The method according to claim 25 or 26 wherein the biological sample is selected from the group including serum, tissue extracts, body fluids, cell culture medium, extracellular medium, supernatants, biopsy specimens or resected tissue.

28. A method according to claim 27 further including adding a dissociating agent to the sample to remove binding proteins.

29. The method according to claim 28 wherein the dissociating agent is selected from the group including SDS, sodium deoxycholate and Tween 20.

30. A composition when used in the method of any one of claims 1-29, said composition comprising an activin β_{C} detection means.

31. The composition according to claim 30 wherein said composition comprises an antibody directed to an epitope of an activin β_{C} subunit together with a suitable diluent, excipient or carrier.

32. The composition according to claim 31 wherein said antibody recognises an epitope of activin β_{C} comprising the amino acid sequence: VPTARRPLSLLYYDRDSNIVKTDIPDMVVEAC (SEQ ID NO:1) or equivalent thereof.

33. A diagnostic kit for use in detecting the onset, predisposition to the onset, or monitoring for the onset or progression of a condition characterised by modulation of the level or bioactivity of activin β_{C} subunit, said kit comprising an activin β_{C} subunit protein and/or encoding nucleic acid detection means in a first compartment and reagents useful for facilitating detection by said detection means in a second compartment.

34. The kit according to claim 33 wherein said detection means is an antibody directed to an epitope of an activin β_{C} subunit together with a suitable diluent, excipient or carrier,

35. The kit according to claim 34 wherein said antibody recognises an epitope of activin β_{C} comprising the amino acid sequence: VPTARRPLSLLYYDRDSNIVKTDIPDMVVEAC (SEQ ID NO:1) or equivalent thereof.

36. The kit according to any one of claims 33-35 when used in the method of any one of claims 1-29.

37. A method of modulating the abnormal growth of a cell, said method comprising modulating the level or bioactivity of activin β_{C} subunit.

38. The method according to claim 37 wherein said abnormal growth is a malignant neoplasm.

39. The method according to claim 38 wherein said malignant neoplasm is a malignant neoplasm of the pancreas, brain and neural tissue, adrenal gland, thyroid gland, stomach, colon, bladder, endometrium, breast, lymph node, skin, salivary gland, bone, nasal cavity, duodenum, gallbaldder, cervix, thymus, larynx, tongue, small intestine, rectum or oesophagus, seminal vesicle cancer, brain cancer, splenic cancer or soft tissue cancer.

40. The method according to claim 37 wherein said abnormal growth is a non-malignant neoplasm.

41. The method according to claim 40 wherein said non-malignant neoplasm is a neoplasm of the fallopian tube, uterus, tonsil, spleen, appendix, seminal vesicle, myometrium or soft tissue.

42. The method according to any one of claims 37-41 wherein up-regulating activin β_{C} subunit levels or bioactivity to a functionally effective level induces said abnormal growth and down-regulating activin β_{C} subunit levels or bioactivity to a functionally ineffective level inhibits said abnormal growth.

43. The method according to claim 42 further comprising administering to said mammal an effective amount of an agent for a time and under conditions sufficient to induce a functionally ineffective level of activin β_{C} subunit.

44. The method according to any one of claims 37-41 wherein down-regulating activin β_{C} subunit levels or bioactivity to a functionally ineffective level induces said abnormal growth and up-regulating activin β_{C} subunit levels or bioactivity to a functionally effective level inhibits said abnormal growth.

45. The method according to claim 44 further comprising administering to said mammal an effective amount of an agent for a time and under conditions sufficient to induce a functionally effective level of activin β_{C} subunit.

46. A method of therapeutically and/or prophylactically treating a condition, or a predisposition to the development of a condition, characterised by an aberrant, unwanted or otherwise inappropriate level or bioactivity of activin β_{C} subunit in a mammal, said method comprising modulating the level of activin β_{C} subunit in said mammal.

47. The method according to claim 46 wherein said activin β_{C} subunit is in monomeric form.

48. The method according to any one of claims 46-47 wherein said condition is a condition of the pancreas, brain and neural tissue, adrenal gland, thyroid gland, stomach, colon, urinary bladder, endometrium, breast, lymph node, skin, salivary gland, bone, nasal cavity, duodenum, gallbladder, uterine cervix, thymus, fallopian tube, uterus, tonsil, spleen, appendix, seminal vesicle, larynx, tongue, small intestine, rectum, oesophagus, myometrium and soft tissue.

49. The method according to claim 48 wherein said condition is a malignant neoplasm.

50. The method according to claim 49 wherein said malignant neoplasm is a malignant neoplasm of the pancreas, brain and neural tissue, adrenal gland, thyroid gland, stomach, colon, bladder, endometrium, breast, lymph node, skin, salivary gland, bone, nasal cavity, duodenum, gallbaldder, cervix, thymus, larynx, tongue, small intestine, rectum or oesophagus, seminal vesicle cancer, brain cancer, splenic cancer or soft tissue cancer.

51. The method according to claim 48 wherein said condition is a non-malignant neoplasm.

52. The method according to claim 51 wherein said non-malignant neoplasm is a neoplasm of the fallopian tube, uterus, tonsil, spleen, appendix, seminal vesicle, myometrium or soft tissue.

53. The method according to any one of claims 46-52 wherein down-regulating said activin β_{C} subunit level to a functionally ineffective level inhibits said abnormal cell growth.

54. The method according to claim 53 said method comprising administering an effective amount of an agent for a time and under conditions sufficient to induce a functionally ineffective level of activin β_{C} subunit.

55. The method according to any one of claims 46-52 wherein up-regulating said activin β_{C} subunit level to a functionally effective level inhibits said abnormal cell growth.

56. The method according to claim 55 said method comprising administering an effective amount of an agent for a time and under conditions sufficient to induce a functionally effective level of activin β_{C} subunit.

57. The method according to claim 54 wherein said agent is an antibody directed to the activin β_{C} subunit.

58. The method according to claim 57 wherein said antibody recognises an epitope of activin β_{C} comprising the amino acid sequence: VPTARRPLSLLYYDRDSNIVKTDIPDMVVEAC (SEQ ID NO:1) or equivalent thereof.

59. Use of an agent capable of modulating the functionally effective level of activin β_{C} subunit in the manufacture of a medicament for the treatment of a condition characterised by an aberrant, unwanted or otherwise inappropriate level of activin β_{C} subunit.

60. Use according to claim 59 wherein said condition is abnormal cell growth.

61. Use according to claim 60 wherein down-regulating activin β_{C} subunit to a functionally ineffective level inhibits said abnormal growth.

62. Use according to claim 60 wherein up-regulating activin β_{C} subunit to a functionally effective level inhibits said abnormal growth.

63. A pharmaceutical composition comprising an agent capable of modulating the functionally effective level of activin β_{C} subunit together with one or more pharmaceutically acceptable carriers and/or diluents.

64. The composition of claim 63 when used in the method of any one of claims 37-58.
